(19) United States
(12) Patent Application Publication
(10) Pub. No.: US 2021/0345587 A1
(43) Pub. Date: Nov. 11, 2021

(54) METHOD FOR OBTAINING CONDITIONED FISH MODELS

(71) Applicants: CENTRE NATIONAL DE LA RECHERCHE SCIENTIFIQUE (CNRS), Paris (FR); UNIVERSITE PARIS-SACLAY, Saint Aubin (FR)

(72) Inventors: Cynthia FROC, Saclay (FR); Kei YAMAMOTO, Palaiseau (FR); Solal BLOCH, Orsay (FR)

(21) Appl. No.: 17/280,735

(22) PCT Filed: Sep. 27, 2019
(86) PCT No.: PCT/EP2019/076177

§ 371 Date: Mar. 26, 2021

(30) Foreign Application Priority Data
Sep. 28, 2018 (FR) 1858985

(51) Int. Cl. A01K 61/10; A61K 49/00; A01K 15/02

(52) U.S. Cl. CPC A01K 61/10; A01K 15/02; A61K 49/0008

(57) ABSTRACT

The present invention concerns a method for obtaining conditioned fish models, and the use of same for characterising a cognitive impairment or deficiency, studying the brain regeneration capacity in a fish model, characterising the working memory and/or the cognitive capacities and/or the learning capacities in a fish model, and identifying molecules, compounds, compositions or formulations modifying the working memory and/or the cognitive capacities and/or the learning capacities in a fish model. The present invention also concerns a device for conditioning a fish model, the device comprising a tank (2) comprising a wall (5) delimiting a cavity (6) capable of containing water for the fish, the cavity (6) comprising a start zone (7) and a selection zone (8), a first partition (3) that can be moved between a closed position and an open position, a first display element (11) for displaying an instruction in the start zone (7), second and third display elements (12, 13) for displaying first and second responses.

### FIELD OF THE INVENTION

The present invention is in the field of animal biology, particularly in the fields of behavioral biology (or ethology) and pathology. It relates to a method for obtaining conditioned model fish. It also relates to the conditioned model fish thus obtained, and to their use to characterize a cognitive impairment or deficiency, resulting for example from neuropsychiatric or neurological disease (such as neurodegenerative disease, addiction or brain injury), particularly with regard to effects on working memory and/or cognitive abilities and/or learning abilities in a model fish; or to study the regenerative capacity of the brain in a model fish; or to characterize working memory and/or cognitive abilities and/or learning abilities in a model fish; or to identify molecules, compounds, compositions or formulations that modify working memory and/or cognitive abilities and/or learning abilities in a model fish. The invention also relates to methods for characterizing a cognitive impairment or deficiency, resulting for example from neuropsychiatric or neurological disease (such as neurodegenerative disease, addiction or brain injury), particularly with regard to effects on working memory and/or cognitive abilities and/or learning abilities in a model fish; or for studying the regenerative capacity of the brain in a model fish. The present invention also relates to a device for conditioning a model fish.

### PRIOR ART

Studies of the etiology, pathogenesis, pathophysiology and semiology of diseases, syndromes or conditions affecting animals in the broadest sense, including humans, as well as the development of therapies, are largely based on studies in laboratory model animals, such as rodents, rabbits, monkeys, etc. Indeed, the use of laboratory animals has made it possible to understand many of the fundamental mechanisms involved in these diseases, syndromes or conditions. It has also allowed the identification, development and testing of a number of therapeutic molecules or formulations and treatment methods.

The use of laboratory animals has played a fundamental role in the understanding of the cognitive abilities of animals, but also of deficiencies, diseases or conditions resulting in a decrease in cognitive abilities (such as neuropsychiatric or neurological disease, including in particular neurodegenerative disease, addiction or brain injury) and the development of effective curative and/or preventive treatments against these diseases.

In this context, laboratory model fish, such as Danio rerio (or zebrafish), have many advantages. Indeed, because of their size (generally smaller than a rodent), their rearing requires little space, which allows more animals to be maintained on a smaller surface. In addition, they are easy and inexpensive to rear. Model fish reproduce easily and in large numbers. Their development is uniform between individuals and rapid (for example zebrafish are considered adult at the age of three months). Some model fish are transparent or can be made transparent with genetic mutations that are currently well understood. Finally, these model fish can be modified by genetic engineering.

Studies of the cognitive abilities of animals and of their modifications in a pathological context generally rely on conditioning of the laboratory animal. Numerous conditioning methods have been described for most model animals, such as rodents, monkeys, or birds. These conditioning methods generally rely on the performance of one or more tasks, more or less complex depending on the species used.

Simple conditioning methods, used in rodents, mammals or birds, are based on the recognition and association of a visual cue with its identical one (task also called “simultaneous matching to sample” or SMTS).

However, such conditioning methods have not been described or implemented with the expected success in fish.

Work carried out on most model fish has shown that they are able to discriminate visual stimuli or cues. For example, discrimination of line color and orientation has been successfully tested in zebrafish (Danio rerio; Cotwill et al., 2005; Arthur and Levin, 2001). Furthermore, it has been shown that the weakly electric fish, Gnathonemus petersii, or the African cichlid (of the genus Pseudotropheus sp.), are able to distinguish geometric shapes (Schuster and Amtsfeld, 2002; Schluessel et al., 2012). It has also been shown that the fish Xenotoca eiseni and the goldfish (Carassius auratus) are able to distinguish subjective and illusory contours (Sovrano and Bisazza, 2009; Wyzisk and Neumeyer, 2007).

However, work aimed at transposing, to fish, the SMTS-type conditioning methods used in rodents, mammals or birds has not been successful. For example, a study conducted on the Malawi cichlid (Pseudotropheus sp.) failed to reach a conclusion on its ability to associate a simple shape with an identical simple shape (Gierszewski et al., 2013).

On the other hand, one study showed that goldfish (Carassius auratus) can perform an SMTS-like task (Goldman and Shapiro, 1979). However, only a small number of individuals achieved and maintained a satisfactory success rate, despite a large number of repetitions, over a period of more than 2 months.

Therefore, there is still a need to develop new and efficient methods of conditioning model fish, for example in order to characterize their cognitive abilities, to study the impact of cognitive impairment or deficiencies, resulting for example from neuropsychiatric or neurological disease (such as neurodegenerative disease, addiction or brain injury) and to identify and develop treatments aiming at preventing, limiting or reversing such cognitive impairment or deficiencies.

The present invention meets this need. The present Inventors have demonstrated for the first time that the zebrafish is capable of performing SMTS-type tasks. The Inventors have thus developed a method for effectively conditioning a model fish to perform such tasks in a reproducible manner. The Inventors have also shown that, surprisingly, the model fish is capable of performing DMTS (“delayed matching to sample”) type tasks, requiring an instruction to be memorized for several seconds. This invention thus allows working memory to be tested, which opens new perspectives for therapeutic modeling. The Inventors have thus revealed for the first time that the model fish can be efficiently conditioned, and this in a deep manner, thanks to the development of novel methods for conditioning a model fish.

### SUMMARY OF THE INVENTION

In the context of the present invention, the Inventors have developed, in a completely unexpected manner, a new method for obtaining conditioned model fish. In particular, this new method makes it possible to obtain conditioned model fish capable of associating an instruction, or stimulus, with a corresponding response.

The Inventors were able to show surprisingly that these conditioned model fish are also able to memorize an instruction for several seconds. In particular, the Inventors have shown that these conditioned model fish are able to associate an instruction with a corresponding response, even after imposing a pause (or delay) time following the removal of the instruction (Bloch et al., 2019). Such abilities had not previously been demonstrated in any model fish.

The present invention therefore relates to a method for obtaining conditioned model fish. The invention also relates to model fish obtainable by the method for obtaining conditioned model fish of the invention.

In the context of the present invention, the Inventors have also developed a new device, particularly adapted to the implementation of the method for obtaining conditioned model fish.

The present invention therefore also relates to a device for conditioning a model fish. Conditioned or deeply conditioned model fish are particularly suitable for studies characterizing the effect of cognitive impairment or deficiencies, resulting for example from neuropsychiatric or neurological disease (such as neurodegenerative disease, addiction or brain injury), on working memory and/or cognitive abilities and/or learning abilities in a model fish; or studies of brain regeneration capacity in a model fish; or studies to identify molecules, compounds, compositions or formulations that modify working memory and/or cognitive abilities and/or learning abilities in a model fish.

The invention thus relates to the use of the model fish conditioned or deeply conditioned to these effects. The invention also relates to methods for characterizing the effect of cognitive impairment or deficiencies, resulting for example from neuropsychiatric or neurological disease (such as neurodegenerative disease, addiction or brain injury), on working memory and/or cognitive abilities and/or learning abilities in a model fish; or methods for studying brain regeneration capacity in a model fish; or methods for identifying molecules, compounds, compositions or formulations that modify working memory and/or cognitive abilities and/or learning abilities in a model fish.

### DESCRIPTION OF THE FIGURES

Other features and advantages will emerge from the following description, which is purely illustrative and non-limiting. The appended figures help to read the parts of the description that refer to them.

FIG. 1 schematically represents, in top view, an example of a device used for the implementation of the method for obtaining a conditioned model fish.

FIGS. 2A and 2B schematically represent the device comprising a first partition in an open and closed position, respectively.

FIG. 3 shows an example of the course of a trial of the pre-conditioning phase of a model fish.

FIG. 4 summarizes an example sequence a conditioning phase trial. ITI denotes “intertrial interval” and corresponds to the period or interval between trials.

FIG. 5 shows an example course of a conditioning phase trial, when the fish chooses a correct response (A and B) or when the fish chooses an incorrect response (C and D). FIG. 5 E summarizes the different possible responses shown in FIG. 5 A to D.

FIG. 6 summarizes an example course of a deep conditioning phase trial.

FIG. 7 shows an example course of a deep conditioning phase trial, when the fish chooses a correct response.

FIG. 8 shows the percentages of correct responses of animal no. 7 during the conditioning phase (or SMTS training; the progression is visualized as a curve) and the deep conditioning phase (or DMTS test/training) with different delays (visualized as dots) in Experiment 1. The threshold of 70% correct responses is indicated by a dotted line.

FIG. 9 shows the percentages of correct responses of animal no. 1 during the conditioning phase (or SMTS test/training; the progression is visualized as a curve) and the deep conditioning phase (or DMTS test/training) with a delay of 4 seconds (visualized as a dot) in Experiment 2. The threshold of 70% correct responses is indicated by a dotted line.

### DETAILED DESCRIPTION OF THE INVENTION

### Definitions

“About equal to” means close, of the same order of magnitude, with no significant difference.

“Fish” means an aquatic vertebrate animal with gills, fins and a body that is generally covered with scales. A fish according to the invention can be a freshwater fish (i.e., a fish whose natural habitat is mainly freshwater), a saltwater fish or a brackish water fish. A fish is understood here as belonging to the taxa Petromyzontidae (lampreys), Chondrichthyes (rays and sharks), Actinopterygii (the most common), Sarcopterygii (Dipnoi and Actinistia). Sometimes the Myxinoidea are associated.

“Model fish” means a fish commonly used in research laboratories for purposes of scientific research. Thus, a model fish is studied in detail to understand a particular biological phenomenon, with the assumption that the results and observations made will be at least partially valid for other organisms, in particularly mammals including humans. Model fishes belong to the genera Danio sp. (for example Danio rerio), Pseudotropheus sp., Gnathonemus sp. (for example Gnathonemus petersii), Pomacentrus sp. (for example Pomacentrus amboinensis), Phoxinus sp. (for example Phoxinus phoxinus), Xenotoca sp. (for example Xenotoca eiseni), Astronotus sp. (for example Astronotus ocellatus), Oryzias sp. (for example Oryzias latipes), Nothobranchius sp. (for example Nothobranchius furzeri), Astyanax sp. (for example Astyanax mexicanus) and Carassius sp. (for example Carassius auratus).

“Test” or “trial” (the two terms being considered synonymous here) means a set of steps carried out successively, comprising the detection of a response selected by a model fish.

“Two consecutive trials” means two trials that are carried out one after the other:
either immediately one after the other, possibly with a pause time (thus according to the sequence “Trial 1-Trial 2” or “Trial 1-Pause-Trial 2”). In this case, the trials are chained one after the other;
or one after the other, observing an interruption time between the two tests (thus according to the sequence “Test 1 - - - interruption - - - Test 2”);

said pause time being generally shorter than said interruption time, as indicated in the following detailed description. The pause between two trials is also referred to as the “intertrial interval” or “ITI”.

“Series” or “session” (the two terms being considered synonymous here) means the repetition of at least two consecutive trials.

“Two consecutive series” or “two consecutive sessions” refers to two series or two sessions that are performed one after the other:
either immediately one after the other, possibly with a pause time (thus according to the sequence “Session 1-Session 2” or “Session 1-Pause-Session 2”);
or one after the other, with an interruption time between the two sessions (thus according to the sequence “Session 1 - - - Interruption - - - Session 2”;

said pause time being generally shorter than said interruption time, as indicated in the following detailed description.

“Pause time” or “pause” refers to a time observed between two consecutive trials or between two consecutive sessions, during which no trial or session is performed. The pause time observed between two consecutive trials (or ITI) is generally (but not systematically) shorter than the pause time observed between two consecutive sessions, as indicated in the following detailed description.

“Interruption” or “interruption time” refers to the time observed between two consecutive trials or between two consecutive sessions, during which no trial or session is performed. The duration of the interruption is usually longer than the pause time.

“Instruction”, “cue” or “sample” means a stimulus, preferably visual, presented to a model fish during a test. The instruction stimulus is an instruction or cue that indicates to the fish the expected response to pass the test, namely at least one response identical to the instruction.

“Response” means a stimulus, preferably visual, presented to a model fish during a test. At least two response stimuli are presented to said model fish, at least one of which is identical to the instruction stimulus and at least one of which is different from the instruction stimulus. In a trial, the fish is expected to choose a response (one response stimulus from the at least two response stimuli presented to it), in a successful trial, the fish chooses a response that is identical to the instruction, in a failed trial, the fish chooses a response that is different from the instruction.

“Correct response” or “good response” thus means a response identical to the instruction, chosen by a model fish during a trial. If the model fish chooses a correct response, then the trial is successful. Advantageously, if the model fish chooses a correct response in the time allotted to it to choose a response during a trial, then the trial is successful.

“Incorrect response” or “wrong response” means a response different from the instruction, chosen by a model fish during a trial, or a failure of the model fish to choose a response within the time allowed to choose a response during a trial. If the model fish chooses an incorrect response, or does not choose any response within the time allowed, then the trial has failed.

“Success rate” means the percentage of correct responses selected/obtained by a model fish per session. The success rate is therefore calculated by adding the total number of correct responses obtained during a trial, for each session.

“Test” or “task” means the set of sessions and trials conducted on the model fish with a specific procedure to assess behavioral parameters. For example, SMTS and DMTS are two separate tests or tasks.

### Method for Obtaining Conditioned Model Fish

The Inventors have developed, in a surprising way, a new method to obtain conditioned model fish. In particular, the Inventors were able to show for the first time that this method makes it possible to obtain conditioned model fish, capable of associating an instruction, or stimulus, with a corresponding response.

The present invention therefore relates to a method for obtaining conditioned model fish, comprising a conditioning phase a); said conditioning phase being characterized in that it consists of at least one session of at least two consecutive trials; each trial comprising the steps consisting in:
(a-i) presenting an instruction to said model fish;
(a-ii) simultaneously presenting at least two distinct responses to the model fish of step (a-i) for a duration Dᵣₐ, said at least two distinct responses comprising at least one response identical to said instruction and at least one response different from said instruction; and
(a-iii) detecting the response chosen by the model fish of step (a-ii) from among said at least two distinct responses, during said duration Dᵣₐ,

said model fish being conditioned when it chooses at least one of said response(s) identical to said instruction with a predetermined success rate T1 per session.

In other words, the conditioned model fish selects at least one of said response(s) identical to said instruction with a predetermined success rate T1 per session.

The method according to the invention is therefore a method for learning, cognitive training and education of a model fish.

In particular, the invention relates to a method for teaching model fish, comprising a conditioning phase a); said conditioning phase being characterized in that it consists of at least one session of at least two consecutive trials; each trial comprising the steps consisting in:
(a-i) presenting an instruction to said model fish;
(a-ii) simultaneously presenting at least two distinct responses to the model fish of step (a-i) for a duration Dᵣₐ, said at least two distinct responses comprising at least one response identical to said instruction and at least one response different from said instruction; and
(a-iii) detecting the response chosen by the model fish of step (a-ii) from among said at least two distinct responses, during said duration Dᵣₐ,

said model fish being conditioned when it chooses at least one of said response(s) identical to said instruction with a predetermined success rate T1 per session.

It is possible to present said instruction during the entire duration of a trial of said conditioning phase a). It is also possible to remove the instruction, either at the same time or during step (a-ii) (for example at the time of presenting the responses of step (a-ii), or during the presentation of the responses of step (a-ii)), or between steps (a-ii) and (a-iii), or after step (a-iii).

According to an embodiment, the instruction of step (a-i) of said conditioning phase a) is presented for a determined duration D_{ca}, and then removed, either at the same time or during step (a-ii) (for example at the time of presenting the responses of step (a-ii), or during the presentation of the responses of step (a-ii)), or between steps (a-ii) and (a-iii). Said determined duration D_{ca} may for example range from 1 second to 5 minutes, preferably from 5 seconds to 4 minutes, preferably from 10 seconds to 3 minutes, preferably from 15 seconds to 2 minutes, preferably from 20 seconds to 1 minute.

According to a preferred embodiment, said determined duration D_{ca} ranges from 1 second to 300 seconds, preferably from 3 seconds to 240 seconds, preferably from 5 seconds to 240 seconds, preferably from 10 seconds to 180 seconds, more preferably from 15 seconds to 120 seconds, preferably from 20 seconds to 100 seconds, preferably from 25 seconds to 90 seconds, preferably from 30 seconds to 60 seconds, more preferably from 6 seconds to 35 seconds, more preferably from 7 seconds to 30 seconds, more preferably from 8 seconds to 25 seconds, more preferably from 9 seconds to 20 seconds, more preferably from 10 seconds to 15 seconds. Preferably, said determined duration D_{ca} is selected from 5, 6, 7, 8, 9, 10, 11, 12, 13, 14, 15, 20, 25, 30, 35, 40, 45, 50, 55, 60, 70, 80, 90, 100, 110, 120, 130, 150, 180, 200, 240 and 300 seconds. Particularly preferably, said determined duration D_{ca} is selected from about 30 seconds, 30 seconds, about 45 seconds, 45 seconds, about 60 seconds, 60 seconds, about 90 seconds, 90 seconds, about 120 seconds, and 120 seconds.

It is also possible to present the responses of step (a-ii) simultaneously with the instruction of step (a-i). In this case, steps (a-i) and (a-ii) are performed simultaneously.

The responses from step (a-ii) are presented to the model fish for a determined duration Dᵣₐ. Said determined duration Dᵣ, may for example range from 1 second to 10 minutes, preferably from 5 seconds to 9 minutes, preferably from 10 seconds to 8 minutes, preferably from 15 seconds to 7 minutes, preferably from 20 seconds to 6 minutes, preferably from 25 seconds to 5 minutes, preferably from 30 to seconds to 4 minutes, preferably from 35 seconds to 3 minutes, preferably from 40 seconds to 2 minutes, preferably from 45 seconds to 60 seconds.

According to a preferred embodiment, said determined duration Dᵣ, ranges from 1 second to 300 seconds, preferably from 3 seconds to 240 seconds, preferably from 5 seconds to 200 seconds, preferably from 10 seconds to 180 seconds, more preferably from 15 seconds to 120 seconds, preferably from 20 seconds to 100 seconds, preferably from 25 seconds to 90 seconds preferably from 30 seconds to 60 seconds, more preferably from 25 seconds to 35 seconds, more preferably from 5 seconds to 90 seconds, more preferably from 10 seconds to 80 seconds, more preferably from 15 seconds to 60 seconds, more preferably from 20 seconds to 50 seconds, more preferably from 25 seconds to 45 seconds, more preferably from 30 seconds to 35 seconds. Particularly preferably, said determined duration Dₐ is selected from 5, 6, 7, 8, 9, 10, 11, 12, 13, 14, 15, 20, 25, 30, 35, 40, 45, 50, 55, 60, 70, 80, 90, 100, 110, 120, 130, 150, 180, 200, 240 and 300 seconds. Preferably, said determined duration Dᵣ, is about 30 seconds or 30 seconds. For a given trial, during the detection step (a-iii), the response chosen by the model fish of step (a-ii) from among said at least two distinct responses, is said to be correct when the choice of at least one of said at least one response identical to the instruction of step (a-i) by the model fish is detected at the expiration of the duration Dᵣₐ. In other words, the response is said to be correct when said model fish chooses at least one of said one or more responses identical to the instruction of step (a-i), during the duration of presentation of the responses Dᵣₐ.

For a given trial, during the detection step (a-iii), the response chosen by the model fish of step (a-ii) from among said at least two distinct responses is said to be incorrect when the choice of at least one of said responses different from the instruction of step (a-i) by the model fish is detected at the expiration of the duration Dᵣₐ. In other words, the response is said to be incorrect when said model fish chooses at least one of said response(s) different from the instruction of step (a-i), during the duration of presentation of the responses Dᵣₐ.

According to an embodiment, for a given trial, the response is also said to be incorrect when no response choice by the model fish is detected at the expiration of the duration Dᵣₐ, during the detection step (a-iii). In other words, according to this embodiment, the response is also said to be incorrect when the model fish has not made any choice during said duration Dᵣₐ, during the detection step (a-iii). According to this embodiment, the response is thus said to be incorrect when said model fish chooses at least one of said response(s) different from the instruction of step (a-i), or when said fish has made no choice, during said duration of presentation of the responses Dᵣₐ. According to this embodiment, said duration Dᵣₐ represents the time given to the model fish to choose a response, during a trial. Said duration Dᵣₐ is as defined above. Advantageously, each trial of the conditioning phase a) comprises the additional step (a-iii′), performed downstream of step (a-iii). Said step (a-iii′) consists in concluding that the response chosen by the model fish of step (a-iii), is the response chosen by the model fish of step (a-iii) (or the absence of response, according to the embodiment described above) at the expiration of the duration Dᵣₐ.

According to an embodiment, each session of the conditioning phase a) comprises from, or consists of, 2 to 20 trials, preferably from 3 to 18 trials, preferably from 4 to 16 trials, preferably from 5 to 15 trials, preferably from 6 to 14 trials, more preferably from 7 to 13 trials, more preferably from 8 to 12 trials, more preferably from 9 to 11 trials, more preferably 10 trials. For a given session, the trials may be chained one after the other.

When the trials are performed in a chained manner, a pause, of a determined duration may be observed between each trial in a session of the conditioning phase a). During this pause, no instructions are presented to the fish and no trials are performed. Preferably, said determined duration ranges from 0.5 to 60 seconds, more preferably from 1 to 30 seconds, more preferably from 2 to 25 seconds, more preferably from 3 to 20 seconds, more preferably from 4 to 15 seconds, more preferably from 5 to 14 seconds, even more preferably from 6 to 13 seconds, still more preferably from 7 to 12 seconds, even more preferentially from 8 to 11 seconds, still more preferentially from 9 to 10 seconds. Preferably, said determined duration of said pause is about 10 seconds, more preferably 10 seconds. The pause between two trials is also referred to as “intertrial interval” or “ITI”.

Alternatively, it is possible to introduce an interruption between each trial, or every two trials or more. During this interruption, no instructions are presented to the fish and no trial is performed. The duration of such an interruption may for example range from several minutes (for example 3, 4, 5, 10, 15, 20, 25, 30, 35, 40, 45, 50 or 55 minutes) to one hour, or from one or more hours (for example 1 to 7 hours, or 8 to 23 h) to one day (i.e., 24 h), or several days (for example 2 to 15 days).

The interruption is generally longer than the pause.

According to a preferred embodiment, each session of the conditioning phase a) comprises, or consists of, a repetition of consecutive trials, the number of which is selected from 5 trials, 6 trials, 7 trials, 8 trials, 9 trials, 10 trials, 11 trials, 12 trials, 13 trials, 14 trials and 15 trials. The Inventors have shown that the number of correct responses selected by the model fish is particularly optimal when a session includes 10 consecutive trials. Also, advantageously, each session of the conditioning phase a) consists of a repetition of 10 consecutive trials. Advantageously, each session of the conditioning phase a) consists of a repetition of 10 consecutive trials performed in a chained manner. Advantageously, each session of the conditioning phase a) consists of a repetition of 10 consecutive trials performed in a chained manner, with the observation of a pause (ITI) as defined above (preferably a pause ranging from 6 to 13 seconds and particularly preferably a pause ranging from 8 to 11 seconds, for example a pause of 10 seconds), between each trial of the session.

According to an embodiment, the conditioning phase a) comprises from, or consists of, 2 to 30 sessions, preferably from 3 to 25 sessions, preferably from 4 to 22 sessions, preferably from 5 to 20 sessions, preferably from 6 to 19 sessions, more preferably from 7 to 18 sessions, more preferably from 8 to 17 sessions, more preferably from 9 to 16 sessions, more preferably from 10 to 15 sessions. The sessions may be performed consecutively (the term “consecutive” being defined in relation to the sessions as indicated above in relation to the trials, i.e., the sessions performed consecutively are either chained sessions, with or without the observation of a pause between each session (the term “pause” being defined in relation to the sessions as indicated above in relation to the trials), or sessions separated by an interruption (the term “interruption” being defined in relation to the sessions as indicated above in relation to the trials)). The pause time observed between two consecutive sessions is generally (but not systematically) longer than the pause time observed between two consecutive trials.

Thus, for a given conditioning phase a), consecutive sessions can be performed in a chained manner one after the other.

When the sessions are performed in a chained manner, a pause, of a determined duration, can be observed between each session of the conditioning phase a). During this pause, no instructions are presented to the fish and no trials are performed. Preferably, said determined duration ranges from 15 to 300 seconds, more preferably from 20 to 240 seconds, more preferably from 25 to 180 seconds, more preferably from 30 to 150 seconds, more preferably from 40 to 120 seconds, more preferably from 45 to 100 seconds, more preferably from 50 to 90 seconds, more preferably from 60 to 80 seconds, more preferably from 65 to 75 seconds. Preferably, said determined duration is 15, 30, 45, 60, 90, 120, 240 or 300 seconds.

Alternatively, it is possible to introduce interruptions between each consecutive session, or every 2 consecutive sessions or more. During this interruption, no instructions are presented to the fish and no trial is performed. The duration of such an interruption can for example range from several minutes (for example 10, 15, 20, 25, 30, 35, 40, 45, 50 or 55 minutes) to one hour, or from one to a few hours (for example from 1 to 7 hours, or 8 to 23 h) to one day (i.e., 24 h) or a few days (for example 2 to 15 days).

As indicated above for testing, the interruption is usually longer than the pause.

According to a preferred embodiment, the conditioning phase a) comprises, or consists of, a repetition of consecutive sessions, the number of which is selected from 5 sessions, 6 sessions, 7 sessions, 8 sessions, 9 sessions, 10 sessions, 11 sessions, 12 sessions, 13 sessions, 14 sessions and 15 sessions. Advantageously, each session of the conditioning phase a) consists of a repetition of 10 consecutive trials.

According to an embodiment, 1 to 10 sessions per day, preferably 1 to 9 sessions per day, more preferably 2 to 8 sessions per day, more preferably 3 to 6 sessions per day, more preferably 4 to 5 sessions per day are performed.

According to a preferred embodiment, 1 session of 10 consecutive trials per day is performed. According to a preferred embodiment, 1 session of 10 consecutive trials per day is performed, with an interruption ranging from one night, to one or more days, between each session (for example an interruption of 10 h to 18 h, of 1 day, of 2 days, of 3 days, of 4 days, of 5 days, of 6 days, of 7 days, of 8 days, of 9 days, of 10 days, of 11 days, of 12 days, of 13 days, 14 days, or 15 to 60 days), over a period ranging from 1 to 90 days, preferably 3 to 60 days, more preferably 5 to 50 days, more preferably 5 to 40 days, more preferably 5 to 30 days, more preferably 5 to 25 days, more preferably 5 to 20 days, more preferably 5 to 15 days, more preferably 5 to 10 days.

“Predetermined success rate T1” means the minimum success rate obtained by a given model fish, per session of the conditioning phase a), from which the model fish is conditioned.

According to an embodiment, said success rate T1 is at least 70%, preferably at least 75%, more preferably at least 80%, more preferably at least 85%, more preferably at least 90%, more preferably at least 95%, more preferably 100%.

According to an embodiment, the model fish is conditioned when it achieves a success rate T1 per session of at least 70%, preferably at least 75%, more preferably at least 80%, more preferably at least 85%, more preferably at least 90%, more preferably at least 95%, more preferably 100%.

According to a preferred embodiment, the model fish is conditioned when it obtains a success rate T1 per session of at least 70%, preferably at least 75%, more preferably at least 80%, more preferably at least 85%, more preferably at least 90%, more preferably at least 95%, more preferably of 100%, for at least two consecutive sessions (chained or not). According to a preferred embodiment, the model fish is conditioned when it obtains a success rate T1 per session of at least 70% for at least two consecutive sessions (chained or not), preferably a success rate T1 per session of at least 70% for at least three consecutive sessions (chained or not), more preferably at least 75%, more preferably at least 80%, more preferably a success rate T1 per session of at least 85%, more preferably at least 90%, more preferably at least 95%, more preferably 100%, for at least three consecutive sessions (chained or not).

According to a preferred embodiment, the model fish is conditioned when it obtains a success rate T1 per session of at least 70% (more preferably at least 75%, more preferably at least 80%, more preferably a success rate T1 per session of at least 85%, more preferably at least 90%, more preferably at least 95%, more preferably at least 100%) for at least three consecutive sessions, each session consisting of or comprising at least one repetition of 10 trials (performed in a chained manner or not). According to a particularly preferred embodiment, the model fish is conditioned when it obtains a success rate T1 per session of at least 70% (more preferably at least 75%, more preferably at least 80%, more preferably a success rate T1 per session of at least 85%, more preferably at least 90%, more preferably at least 95%, more preferably at least 100%) for at least three consecutive sessions, each session consisting of a repetition of 10 trials performed in a chained manner.

According to an embodiment, the model fish is conditioned when it obtains a success rate T1 per session of at least 70% (more preferably at least 75%, more preferably at least 80%, more preferably a success rate T1 per session of at least 85%, more preferably at least 90%) for at least two consecutive sessions (chained or not), said success rate T1 of at least 70% for at least two consecutive sessions being preferably reached as early as the fifteenth session performed (chained or not), more preferably as early as the twelfth session performed (chained or not), more preferably as early as the eleventh session performed (chained or not), more preferably as early as the tenth session performed (chained or not), more preferably as early as the ninth session performed (chained or not), more preferably as early as the eighth session performed (chained or not). In this case, each session preferably consists of or preferably comprises at least one repetition of 10 trials (performed in a chained manner or not).

According to a preferred embodiment, the model fish is conditioned when it obtains a success rate T1 per session of at least 70% (more preferably at least 75%, more preferably at least 80%, more preferably a success rate T1 per session of at least 85%, more preferably at least 90%) for at least three consecutive sessions (chained or not), said success rate T1 of at least 70% for at least three consecutive sessions being preferably reached as early as the fifteenth session performed (chained or not), more preferably as early as the twelfth session performed (chained or not), more preferably as early as the eleventh session performed (chained or not), more preferably as early as the tenth session performed (chained or not), more preferably as early as the ninth session performed (chained or not). In this case, each session preferably consists of, or preferably comprises, at least one repetition of 10 trials (performed in a chained manner or not).

According to a particularly preferred embodiment, the model fish is conditioned when it obtains a success rate T1 per session of at least 70% (more preferably at least 75%, more preferably at least 80%, more preferably a success rate T1 per session of at least 85%, more preferably at least 90%) for at least three consecutive sessions (chained or not), said success rate T1 of at least 70% for at least three consecutive sessions being preferably reached as early as the fifteenth session performed (chained or not), more preferably as early as the twelfth session performed (chained or not), more preferably as early as the eleventh session performed (chained or not), more preferably as early as the tenth session performed (chained or not), more preferably as early as the ninth session performed (chained or not), each session consisting of a repetition of 10 trials performed in a chained manner.

The method for obtaining conditioned model fish can be carried out simultaneously/in parallel on a group of individuals, said individuals belonging to the same genus of model fish or not, to the same species of model fish or not. Said group consists of a predetermined number of model fish, comprising at least three individuals. For example, said group consists of 3, 4, 5, 6, 7, 8, 9, 10, 12, 15 model fish. Said predetermined number of individuals ranges for example from ten or so to several hundred model fish. Preferably, the group of individuals is evaluated as a whole.

Thus, according to a first embodiment, the group of model fish is conditioned when a predetermined number of individuals obtain a predetermined success rate T1 as defined above. The group of model fish is conditioned when at least half (50%) of the individuals of the group obtain a predetermined success rate T1 as defined above. Preferably, the group of model fish is conditioned when at least 55% of the individuals of the group obtain a predetermined success rate T1 as defined above, more preferably when at least 60% of the individuals of the group, more preferably when at least 65% of the individuals of the group, more preferably when at least 70% of the individuals of the group, more preferably when at least 75% of the individuals of the group, more preferably when at least 80% of the individuals of the group, more preferably when at least 85% of the individuals of the group, more preferably when at least 90% of the individuals of the group, more preferably when at least 95% of the individuals of the group, more preferably when 100% of the individuals of the group, obtains a predetermined success rate T1 as defined above.

According to an embodiment, said instruction and/or said at least one response is a visual stimulus. For example, the visual stimulus is selected from a color, a geometric shape (in 2 or 3 dimensions), a non-geometric shape (in 2 or 3 dimensions), an image (in 2 or 3 dimensions), an object (in 2 or 3 dimensions) a light signal or any combination thereof (such as a light signal in the form of a color, a geometric shape (in 2 or 3 dimensions), a non-geometric shape (in 2 or 3 dimensions), an image (in 2 or 3 dimensions), or an object (in 2 or 3 dimensions)). When the visual stimulus consists of or includes a light signal, said light signal is emitted by a light source, for example a primary light source, such as a (electric) lamp, a light emitting diode (or LED), a laser, or a screen. The visual stimulus may comprise any natural or synthetic material, such as paper, cardboard, glass, metal, fabric, or any mixture of these materials. The visual stimulus may for example be represented on a sheet of paper, a card, a photograph, a drawing. The visual stimulus may be displayed or projected on a screen. According to an embodiment, said instruction and/or said at least one response is a color.

Preferably, the visual stimulus is located in the visible spectrum (thus having a vacuum wavelength ranging from about 380 to about 780 nanometers (nm)), or in the ultraviolet (thus having a vacuum wavelength ranging from about 400 to 100 nm).

According to an embodiment, the step of detecting (a-iii) the response chosen by the model fish comprises detecting the passage and/or presence of said model fish in a response zone (or choice zone). Said response zone (or choice zone) corresponds to a space or sector in which a response is presented. In the case of a visual stimulus, said response zone corresponds to a space or sector in which said visual stimulus is presented. The detection of the presence of the model fish in a response zone (or choice zone) comprises, or consists of, for example, the detection of the passage (or crossing) of said model fish through a line (or border, limit, or boundary) of demarcation of said response zone, or a line (or border, limit, or boundary) located inside the response zone. Thus, said line is a line dedicated to the detection of the response chosen by the fish. Said line is therefore called a “response line”. Such a line makes it possible to discriminate the response chosen by the fish. Thus, the fish chooses a response when it passes through said response line. In other words, when the fish passes through said response line, the choice of the fish is validated (validly detected). In other words, the detection of the passage of the fish through said response line corresponds to the detection of the response chosen by the fish. The person skilled in the art will easily understand that it may be sufficient for the fish to pass a portion of its body through said response line for its passage through the response line to be detected. For example, it may be sufficient for the fish to pass its head through the response line for its passage through the line to be detected. Preferably, the passage of the fish through the response line is detected when the fish passes entirely (i.e., its entire body) through the line.

Said response line can be virtual or not. The response line may be marked or drawn by a visible line or a long object (movable or not), or marked with a light signal. Alternatively, said response line may comprise, or consist of, a partition or wall (movable or not). Said response line may for example comprise or consist of a partition or wall separating a start zone, in which the instruction is presented, from a response zone (or choice zone), in which at least one response is presented.

Alternatively or in combination, said response line (and/or said response zone or choice zone) consists of, or comprises, an orifice (or an opening, a hole or an recess, a cavity or a niche). In this case, the orifice is an orifice dedicated to the detection of the response chosen by the fish. This orifice is therefore called a “response orifice”. Such an orifice makes it possible to discriminate the response chosen by the fish. Thus, the fish chooses a response when it passes through said response orifice. In other words, when the fish passes through said response orifice, the choice of the fish is validated (validly detected). In other words, the detection of the passage of the fish through said response orifice corresponds to the detection of the response chosen by the fish. Said response orifice is for example an orifice of a reward dispenser. Said response orifice may be an orifice in a wall, such as a window, an opening, a hole, or an recess, a cavity or a recess. In this case, the response orifice may for example be a hole in a wall separating a start zone, in which the instruction is presented, and a choice zone, in which at least one response is presented. The skilled person will easily understand that it may be sufficient for the fish to pass a portion of its body through the response orifice for its passage through the orifice to be detected. For example, in the case of an orifice of a reward dispenser, it may be sufficient for the fish to pass its head through said orifice, so as to take its reward, for its passage through the response orifice to be detected.

Advantageously, the step of detecting (a-iii) the response chosen by the model fish is performed by means of sensors. When the detection of the response chosen by the model fish comprises the detection of the passage of said model fish in a response zone as defined above, for example the detection of its passage through a response line as defined above (for example an orifice as defined above), at least one sensor is positioned so as to detect the passage of the fish in said response zone, for example through said response line (for example in said response orifice). Alternatively or in combination, the detection of the response chosen by the model fish (for example the detection of the passage of said model fish through a response zone) is visual, for example a detection directly by the experimenter.

It may be advantageous to detect the passage of the fish through a line that is not dedicated to the detection of the response chosen by the fish (for example the passage of the fish through an orifice that is not dedicated to the detection of the response chosen by the fish). In particular, such a detection allows the fish to be tracked during the course of a trial and/or a session and or a test. Said line which is not dedicated to the detection of the response chosen by the fish is therefore called a “tracking line” (or “monitoring” line). Said tracking line is defined as the response line. Said tracking line comprises for example, or consists of, an orifice, called a “tracking orifice” (or “monitoring orifice”). Said tracking orifice is for example a hole in a wall, such as a window, an opening, a hole or even a recess, a cavity or a niche. In this case, the tracking orifice may for example be a hole in a wall separating a start zone, in which the instruction is presented, and a choice zone, in which the at least two responses are presented. In this case, detection of the passage of the fish through said tracking orifice indicates that the fish is exiting the start zone and is preparing to choose (or has already chosen) a response. Advantageously, the detection of the passage of the fish through a tracking orifice is performed by means of sensors. Alternatively or in combination, the detection of the passage of the fish through a tracking orifice is visual, for example a detection directly by the experimenter.

Alternatively or in combination, it may be advantageous to detect the movements and/or position of the model fish during all or part of a trial and/or a session and/or the conditioning phase a). In particular, such detection allows the tracking (or monitoring) of the fish during the course of all or part of a trial and/or a session and/or the conditioning phase a). The tracking detection of the fish is for example carried out by means of sensors (for example by means of several sensors positioned in different zones (start zone, response zone), motion tracking camera(s), and/or a “tracking” or “monitoring” system. Alternatively or in combination, the detection can be visual, for example a detection directly by the experimenter.

According to an embodiment, the conditioning phase a) further comprises an additional step performed downstream of the detection step (a-iii). Said additional step preferably comprises:
dispensing a reward to said model fish when it has chosen at least one of said response(s) identical to said instruction during said duration Dᵣₐ, said reward preferably comprising food suitable for said model fish; or
not dispensing a reward to said model fish when it has chosen at least one of said response(s) different from said instruction during said duration Dᵣ, and, optionally, isolating and/or darkening said model fish from said model fish when it has chosen at least one of said response(s) different from said instruction (or optionally, when it has chosen no response) during said duration Dᵣₐ,

The present invention therefore relates to a method for obtaining conditioned model fish, comprising a conditioning phase a); said conditioning phase being characterized in that it consists of at least one session of at least two consecutive trials; each trial comprising the steps consisting in:
(a-i) presenting an instruction to said model fish;
(a-ii) simultaneously presenting at least two distinct responses to the model fish of step (a-i) for a duration Dᵣₐ, said at least two distinct responses comprising at least one response identical to said instruction and at least one response different from said instruction;
(a-iii) detecting the response chosen by the model fish of step (a-ii) from among said at least two distinct responses, during said duration Dᵣₐ; and
(a-iv) dispensing a reward to said model fish when it has chosen at least one of said response(s) identical to said instruction during said duration Dᵣₐ, said reward preferably comprising food suitable for said model fish; or
not dispensing a reward to said model fish when it has chosen at least one of said response(s) different from said instruction during said duration Dᵣₐ and, optionally, isolating and/or darkening said model fish when it has chosen at least one of said response(s) different from said instruction (or optionally, when it has chosen no response) during said duration Dᵣₐ.

said model fish being conditioned when it chooses at least one of said response(s) identical to said instruction with a predetermined success rate T1 per session.

Advantageously, said reward is dispensed by a reward dispenser. For example, the dispensing of said reward is triggered by the passage and/or detection of the presence of said model fish in a response zone, for example through a response line (comprising or consisting of a response orifice). Advantageously, said response zone, said response line and/or said response orifice is as defined above. In particular, said response line and/or said response orifice is dedicated to the detection of the response chosen by the fish. In particular, said response orifice may be an orifice of a reward dispenser or an orifice in a wall, such as a window, an opening, a hole or a recess, a cavity or a niche.

Advantageously, said reward is distributed to the model fish only when it has chosen at least one correct response during said duration Dᵣₐ. In this case, the model fish does not receive a reward when it has chosen at least one incorrect response during said duration Dᵣₐ. The correct response and the incorrect response are as defined above. In particular, according to an embodiment, for a given trial, the response is also said to be incorrect when no response choice by the model fish is detected at the expiration of the duration Dᵣₐ, during the detection step (a-iii). In other words, according to this embodiment, the response is also said to be incorrect when the model fish has not made any choice during said duration Dᵣₐ, during the detection step (a-iii). According to this embodiment, the response is thus said to be incorrect when said model fish chooses at least one of said response(s) different from the instruction of step (a-i), or when said fish has made no choice, during said duration of presentation of the responses Dᵣₐ. According to this embodiment, said duration Dᵣ, represents the time given to the model fish to choose a response, during a trial. Said duration Dᵣ, is as defined above.

According to an embodiment, the model fish is isolated when it has chosen at least one incorrect response during said duration Dᵣ, (and/or when the fish has made no choice during said duration Dᵣₐ, according to the embodiment). According to this isolation step, said model fish can for example be isolated (or enclosed, or confined) in a defined zone, for a determined duration Advantageously, said determined duration Dᵢ during which the fish is isolated ranges from 5 seconds to 60 seconds, preferably from 10 seconds to 50 seconds, more preferably from 20 seconds to 40 seconds, more preferably from 25 seconds to 35 seconds, more preferably, said duration is selected from 10 seconds, 15 seconds, 20 seconds, 25 seconds, 30 seconds, 35 seconds, 40 seconds, and 45 seconds, and particularly preferably from about 30 seconds or 30 seconds. The model fish is for example isolated in the start zone as defined above. Alternatively or in combination, the model fish is placed in darkness when it has chosen at least one incorrect response during said duration Dᵣ, (and/or when the fish has made no choice during said duration Dᵣₐ, according to the embodiment), for said determined duration Dᵢ.

According to an embodiment, said reward comprises or consists of feed (or food) suitable for said model fish. For example, the reward comprises or consists of a dried and compacted feed (or food) granule or pellet or flake, such as commercially available granules, pellets or flakes (for example pellets of the Gemma® type, marketed by SKRETTING, such as Gemma Micro® (in particular Gemma Micro® 600)). For example, the reward comprises or consists of insect larva, such as mosquito, fly or gnat larva, said larva being for example fresh, dried, freeze-dried, frozen or deep-frozen. The reward may also comprise or consist of a mixture of food granules, pellets or flakes and insect larvae (mosquito, fly, gnat etc.). The skilled person will be able to define the food adapted to the model fish used.

The Inventors have shown, advantageously, that the number of correct responses selected by the model fish is particularly optimal when the reward is palatable feed (or a palatable food). Advantageously, the reward comprises, or consists of, palatable feed (or a palatable food).

“Palatable feed” or “palatable food” means here feed or food that will attract a model fish to consume it. Thus, a model fish will be preferentially attracted to a palatable feed or a palatable food to consume it. In other words, the feed or food is palatable to a model fish if the model fish shows a preference for said feed or food. For example, in the presence of several different types of feed or several different foods, a model fish will select (prefer) the most palatable feed or food. This attraction (or preference) may come from any of the model fish's senses, but is usually related to, among other things, appearance, smell, taste, aroma, flavor, texture, and/or mouthfeel. For example, palatable feed or food that is palatable to a model fish is feed or food that visually and/or olfactory and/or gustatory resembles feed consumed (or food consumed) by the model fish in its natural environment.

The Inventors have also shown that, advantageously, the number of correct responses chosen by the model fish was particularly optimal when the reward is a nutritionally poor feed or food. Advantageously, the reward comprises, or consists of, nutritionally poor feed or food. More advantageously, the reward comprises, or consists of, palatable and nutritionally poor feed or a palatable and nutritionally poor food.

“Nutritionally poor feed” or “depleted feed” or “nutritionally poor or depleted feed”, “nutritionally poor food” or “depleted food” or “nutritionally poor or depleted food” means a dose (or ration) of a feed or food suitable for the model fish used, providing less than 20%, preferably less than 15%, more preferably less than 10%, more preferably less than 5%, more preferably less than 3%, of the average daily amount of fat, and/or protein, and/or carbohydrate required by said adult model fish.

Examples of palatable and/or nutritionally poor food or feed for model fish include freeze-dried insect larvae, such as freeze-dried mosquito, fly or gnat larvae. Freeze-dried insect larvae are commercially available, such as freeze-dried insect larvae marketed by the firm JBL, for example the NovoFil® line of larvae. Examples of nutritionally poor food or feed for model fish comprise dried and compacted food granules, pellets or flakes with a poor or depleted fat, and/or protein, and/or carbohydrate content (for example a pellet that provides less than 20%, preferably less than 15%, more preferably less than 10%, more preferably less than 5%, more preferably less than 3%, of the average daily amount of fat, and/or protein, and/or carbohydrate, required for an adult model fish). In particular, the Inventors have shown that the success rate T1 is particularly optimal when the reward comprises freeze-dried insect larva. The Inventors have also shown that a success rate T1 of at least 70% was achieved more quickly (i.e., a success rate T1 of at least 70% was achieved after performing a smaller number of consecutive sessions), when the reward comprises insect larva. Advantageously, the reward comprises or consists of freeze-dried insect larva, such as freeze-dried mosquito, fly or gnat larva, or a mixture of freeze-dried mosquito and/or fly and/or gnat larva. Preferably, the reward comprises or consists of freeze-dried mosquito larva.

According to an embodiment, said model fish is selected from model fish belonging to the genera Danio sp., Pseudotropheus sp., Gnathonemus sp., Pomacentrus sp., Phoxinus sp., Xenotoca sp., Astronotus sp., Oryzias sp., Nothobranchius sp, Astyanax sp. and Carassius sp. preferably from Danio rerio, Metriaclima zebra, Gnathonemus petersii, Pomacentrus amboinensis, Phoxinus phoxinus, Xenotoca eiseni, Astronotus ocellatus, Oryzias latipes, Nothobranchius furzeri, Astyanax mexicanus and Carassius auratus. According to an embodiment, the method of the invention further comprises a pre-conditioning (or pre-learning, or pre-education, or pre-training) phase pre-a), carried out upstream of the conditioning phase a). Advantageously, said pre-conditioning phase comprises a step of adapting the model fish to the environment in which the conditioning phase a) and/or the deep conditioning phase b) will take place. The skilled person knows how to determine the steps comprised in said pre-conditioning phase.

For example, said pre-conditioning phase pre-a) comprises a step or steps of adapting the model fish to the device as defined below. For example, in this case, said adaptation step consists of, or comprises, at least one session of at least two trials;

each trial, comprising or consisting of rewarding said model fish if it approaches the response zone of said device to a determined maximum distance, in the absence of any instruction or response, wherein said determined maximum distance decreases with each new trial.

Advantageously, said model fish is pre-conditioned when the model fish is familiar with its environment. The fish is then able to generate a clear behavioral response. In other words, the fish is pre-conditioned/prepared for the implementation of the conditioning/learning method described above, in a given environment.

Said pre-conditioning phase pre-a) comprises, for example, a step of adapting the model fish to a food dispenser that will be used during the conditioning phase a) and/or the deep conditioning phase b) (see the following section).

For example, in this case, the pre-conditioning (or pre-learning, or pre-education, or pre-training) phase pre-a), consists of, or comprises, at least one session of at least two consecutive trials;

each trial comprising the steps consisting in:
(pre-a-i) presenting a reward in a reward dispenser to said model fish;
(pre-a-ii) detecting the taking of the reward from step (pre-a-i) by said fish in the reward dispenser, for example by means of sensors;

said model fish being pre-conditioned when it chooses at least one of said response(s) identical to said instruction with a predetermined success rate T3 per session.

A trial is successful when the model fish takes the reward from the reward dispenser.

“Predetermined success rate T3” means the minimum success rate obtained by a given model fish, per session of the pre-conditioning phase pre-a), from which the model fish is pre-conditioned. Advantageously, the success rate T3 is defined in relation to the pre-conditioning phase pre-a) as previously indicated in relation to the conditioning phase a). Thus, according to an embodiment, said success rate T3 is at least 70%, preferably at least 75%, more preferably at least 80%, more preferably at least 85%, more preferably at least 90%, more preferably at least 95%, more preferably 100%. According to a preferred embodiment, the model fish is pre-conditioned when it achieves a success rate T3 per session of at least 70%, preferably at least 75%, more preferably at least 80%, more preferably at least 85%, more preferably at least 90%, more preferably at least 95%, more preferably 100%.

The instruction, response, trial, number of trials, session, number of sessions, reward, different durations (for example pause duration and interruption duration), detection step and detection, and model fish of the pre-conditioning phase pre-a), are as defined above for the conditioning phase a). In particular, the trials of the pre-conditioning phase pre-a) may be performed in a chained manner or not; the sessions of the pre-conditioning phase pre-a) may be performed consecutively, in a chained manner or not; as defined above in relation to the conditioning phase a).

The present invention also relates to a conditioned model fish obtainable by the method according to the invention as described above.

### Method for Obtaining Deeply Conditioned Model Fish

The Inventors were able to show in a surprising way that the model fish conditioned according to the conditioning phase a) of the method of the invention, are also capable of memorizing an instruction for several seconds. In particular, the Inventors have shown that these conditioned model fish are capable of associating an instruction with a corresponding response, even after having imposed a delay following the removal of the instruction. Such abilities had never before been demonstrated in model fish. The Inventors have thus developed, in a quite surprising way, the first method for obtaining deeply conditioned model fish. Such a method, allowing such capacities in model fish to be obtained, had never before been described.

Thus, the invention further relates to a method for obtaining deeply conditioned model fish, comprising a deep conditioning phase b) characterized in that it consists of at least one session of at least two consecutive trials; each trial comprising the steps consisting in:
(b-i) presenting an instruction to said conditioned model fish for a specified duration;
(b-ii) removing said instruction and then observing a delay of a predetermined duration D1, during which no instruction is presented to the model fish of step (b-i);
(b-iii) simultaneously presenting at least two distinct responses to the model fish of step (b-ii) for a duration D_{rb}, said at least two distinct responses comprising at least one response identical to said instruction and at least one response different from said instruction; and
(b-iv) detecting the response chosen by the model fish of step (b-iii) among said at least two distinct responses, during said duration D_{rb};

said model fish being deeply conditioned when it chooses at least one of said one or more identical responses to said instruction with a predetermined success rate T2 per session.

In other words, the deeply conditioned model fish selects at least one of said response(s) identical to said instruction with a predetermined success rate T2 per session.

The method according to the invention is thus a method of deep learning, deep cognitive training, or deep education, of a model fish.

Thus, according to an embodiment, the method for obtaining a conditioned model fish according to the invention further comprises a deep conditioning phase b) characterized in that it consists of at least one session of at least two consecutive trials; each trial comprising the steps consisting in:
(b-i) presenting an instruction to said conditioned model fish for a specified duration;
(b-ii) removing said instruction and then observing a delay of a predetermined duration D1, during which no instruction is presented to the model fish of step (b-i);
(b-iii) simultaneously presenting at least two distinct responses to the model fish of step (b-ii) for a duration D_{rb}, said at least two distinct responses comprising at least one response identical to said instruction and at least one response different from said instruction; and
(b-iv) detecting the response chosen by the model fish of step (b-iii) among said at least two distinct responses, during said duration D_{rb};

said model fish being deeply conditioned when it chooses at least one of said one or more identical responses to said instruction with a predetermined success rate T2 per session.

The present invention therefore relates to a method for obtaining conditioned model fish, comprising a conditioning phase a); said conditioning phase being characterized in that it consists of at least one session of at least two consecutive trials; each trial comprising the steps consisting in:
(a-i) presenting an instruction to said model fish;
(a-ii) simultaneously presenting at least two distinct responses to the model fish of step (a-i) for a duration Dᵣₐ, said at least two distinct responses comprising at least one response identical to said instruction and at least one response different from said instruction; and
(a-iii) detecting the response chosen by the model fish of step (a-ii) from among said at least two distinct responses, during said duration Dᵣₐ,

said model fish being conditioned when it chooses at least one of said response(s) identical to said instruction with a predetermined success rate T1 per session;

said conditioning phase a) being followed by a deep conditioning phase b) characterized in that it consists of at least one session of at least two consecutive trials; each trial comprising the steps consisting in:
(b-i) presenting an instruction to said conditioned model fish for a specified duration;
(b-ii) removing said instruction and then observing a delay of a predetermined duration D1, during which no instruction is presented to the model fish of step (b-i);
(b-iii) simultaneously presenting at least two distinct responses to the model fish of step (b-ii) for a duration D_{rb}, said at least two distinct responses comprising at least one response identical to said instruction and at least one response different from said instruction; and
(b-iv) detecting the response chosen by the model fish of step (b-iii) among said at least two distinct responses, during said duration D_{rb};

said model fish being deeply conditioned when it chooses at least one of said one or more identical responses to said instruction with a predetermined success rate T2 per session.

According to an embodiment, the instruction of step (b-i) of said deep conditioning phase b) is presented for a determined duration Dₐb, before being removed during step (b-ii). Said determined duration D_{cb} may for example range from 1 second to 5 minutes, preferably from 5 seconds to 4 minutes, preferably from 10 seconds to 3 minutes, preferably from 15 seconds to 2 minutes, preferably from 20 seconds to 1 minute.

According to a preferred embodiment, said determined duration D_{cb} ranges from 1 second to 300 seconds, preferably from 3 seconds to 240 seconds, preferably from 5 seconds to 240 seconds, preferably from 10 seconds to 180 seconds, more preferably from 15 seconds to 120 seconds, preferably from 20 seconds to 100 seconds, preferably from 25 seconds to 90 seconds, preferably from 30 seconds to 60 seconds, more preferably from 6 seconds to 35 seconds, more preferably from 7 seconds to 30 seconds, more preferably from 8 seconds to 25 seconds, more preferably from 9 seconds to 20 seconds, more preferably from 10 seconds to 15 seconds. Particularly preferably, said determined duration D_{cb} is selected from 5, 6, 7, 8, 9, 10, 11, 12, 13, 14, 15, 20, 25, 30, 35, 40, 45, 50, 55, 60, 70, 80, 90, 100, 110, 120, 130, 150, 180, 200, 240 and 300 seconds. Particularly preferably, said determined duration D_{cb} is selected from about 30 seconds, 30 seconds, about 45 seconds, 45 seconds, about 60 seconds, 60 seconds, about 90 seconds, 90 seconds, about 120 seconds, and 120 seconds.

According to an embodiment, the duration D_{cb} of the deep conditioning phase b) is equal to, or about equal to, the duration D_{ca} of the conditioning phase a). According to an alternative embodiment, the duration D_{cb} of the deep conditioning phase b) is different from the duration D_{ca} of the conditioning phase a).

According to an embodiment, the predetermined duration D1 of the delay of step (b-ii) of the deep conditioning phase b) ranges from 0.5 to 10 seconds, preferably from 0.7 to 9 seconds, more preferably from 0.9 to 8 seconds, more preferably from 1 to 7 seconds, more preferably from 1.5 to 6 seconds, more preferably from 2 to 5 seconds, more preferably from 2.5 to 4.5 seconds, more preferably from 3 to 4 seconds. Particularly preferably, said predetermined duration D1 is selected from 3 seconds, about 3 seconds, 3.5 seconds, about 3.5 seconds, 4 seconds, about 4 seconds, 4.5 seconds, about 4.5 seconds, 5 seconds and about 5 seconds, 6 seconds and about 6 seconds. Advantageously, the predetermined duration D1 represents the working memory capacity of the model fish.

The responses of step (b-iii) are presented for a determined duration D_{rb}. Said determined duration D_{rb} may for example range from 1 second to 10 minutes, preferably from 5 seconds to 9 minutes, preferably from 10 seconds to 8 minutes, preferably from 15 seconds to 7 minutes, preferably from 20 seconds to 6 minutes, preferably from 25 seconds to 5 minutes, preferably from 30 to seconds to 4 minutes, preferably from 35 seconds to 3 minutes, preferably from 40 seconds to 2 minutes, preferably from 45 seconds to 60 seconds.

According to a preferred embodiment, said determined duration D_{rb} ranges from 1 second to 300 seconds, preferably from 3 seconds to 240 seconds, preferably from 5 seconds to 240 seconds, preferably from 10 seconds to 180 seconds, more preferably from 15 seconds to 120 seconds, preferably from 20 seconds to 100 seconds, preferably from 25 seconds to 90 seconds, preferably from 30 seconds to 60 seconds, more preferably from 5 seconds to 90 seconds, more preferably from 10 seconds to 80 seconds, more preferably from 15 seconds to 60 seconds, more preferably from 20 seconds to 50 seconds, more preferably from 25 seconds to 45 seconds, more preferably from 30 seconds to 35 seconds. Particularly preferably, said determined duration D_{rb} is selected from 5, 6, 7, 8, 9, 10, 11, 12, 13, 14, 15, 20, 25, 30, 35, 40, 45, 50, 55, 60, 70, 80, 90, 100, 110, 120, 130, 150, 180, 200, 240 and 300 seconds. Preferably, said determined duration D_{rb} is about 30 seconds or 30 seconds.

For a given trial, during the detection step (b-iv), the response chosen by the model fish of step (b-iii) from among said at least two distinct responses is said to be correct when the choice of at least one of said response(s) identical to the instruction of step (b-i) by the model fish is detected at the expiration of the duration D_{rb}. In other words, the response is said to be correct when said model fish chooses at least one of said one or more responses identical to the instruction of step (b-i), during the duration of presentation of the responses D_{rb}.

For a given trial, during the detection step (b-iv), the response chosen by the model fish of step (b-iii) from among said at least two distinct responses is said to be incorrect when the choice of at least one of said responses different from the instruction of step (b-i) by the model fish is detected at the expiration of the duration D_{rb}. In other words, the response is said to be incorrect when said model fish chooses at least one of said response(s) different from the instruction of step (b-i), during the duration of presentation of the responses D_{rb}.

According to an embodiment, for a given trial, the response is also said to be incorrect when no response choice by the model fish is detected at the expiration of the duration D_{rb}, during the detection step (b-iv). In other words, according to this embodiment, the response is also said to be incorrect when the model fish has not made any choice during said duration D_{rb}, during the detection step (b-iv). According to this embodiment, the response is thus said to be incorrect when said model fish chooses at least one of said response(s) different from the instruction of step (b-i), or when said fish has made no choice, during said duration of presentation of the responses D_{rb}. According to this embodiment, said duration D_{rb} represents the time allotted to the model fish to choose a response, during a trial. Said duration D_{rb} is as defined above.

Advantageously, each trial of the deep conditioning phase b) comprises the additional step (b-iv′), performed downstream of step (b-iv). Said step (b-iv′) consists in concluding that the response chosen by the model fish of step (b-iv), is the response chosen by the model fish of step (b-iv) (or the absence of response, according to the embodiment described above) at the expiration of the duration D_{rb}.

Steps b-i), b-iii), and b-iv) of the deep conditioning phase b) are consistent with steps a-i), a-ii), and a-iii) of the conditioning phase a), respectively. Thus, the instruction, response, trial, number of trials, session, number of sessions, reward, different durations (for example pause duration and interruption duration), detection step and detection, and model fish of the deep conditioning phase b), are as defined above for conditioning phase a). In particular, the trials of the deep conditioning phase b) may be performed in a chained manner or not; the sessions of the deep conditioning phase b) may be performed consecutively, in a chained manner or not; as defined above in relation to the conditioning phase a). In the case of the deep conditioning phase b), only one session can be performed. Thus, according to an embodiment, the deep conditioning phase b) comprises, or consists of, a single session (comprising, or consisting of, a repetition of a number of trials, performed in a chained manner or not, as defined above in relation to the conditioning phase a)).

According to an embodiment, the instruction used in step b-i) of the deep conditioning phase b) is identical to the instruction used in step a-i) of the conditioning phase a); and/or the response(s) used in step b-iii) of the deep conditioning phase b) is/are identical to the response(s) used in step a-ii) of the conditioning phase a).

According to an alternative embodiment, the instruction used in step b-i) of the deep conditioning phase b) is different from the instruction used in step a-i) of the conditioning phase a); and/or the response(s) used in step b-iii) of the deep conditioning phase b) is/are different from the response(s) used in step a-ii) of the conditioning phase a).

“Predetermined success rate T2” means the minimum success rate achieved by a given model fish per session of the deep conditioning phase b), from which the model fish is deeply conditioned.

According to an embodiment, said success rate T2 is at least 70%, preferably at least 75%, more preferably at least 80%, more preferably at least 85%, more preferably at least 90%, more preferably at least 95%, more preferably 100%.

According to an embodiment, the model fish is deeply conditioned, when it achieves a success rate T2 per session of at least 70%, preferably at least 75%, more preferably at least 80%, more preferably at least 85%, more preferably at least 90%, more preferably at least 95%, more preferably 100%.

According to a preferred embodiment, the model fish is deeply conditioned when it achieves a success rate T2 per session of at least 70%, preferably at least 75%, more preferably at least 80%, more preferably at least 85%, more preferably at least 90%, more preferably at least 95%, more preferably 100%, for at least two consecutive sessions (chained or not). According to a preferred embodiment, the model fish is deeply conditioned when it achieves a success rate T2 per session of at least 70% for at least two consecutive sessions (chained or not). According to a preferred embodiment, the model fish is deeply conditioned when it achieves a success rate T2 per session of at least 70% (more preferably at least 75%, more preferably at least 80%, more preferably at least 85%, more preferably at least 90%, more preferably at least 95%, more preferably at least 100%) for at least three consecutive sessions (chained or not). According to a preferred embodiment, the model fish is deeply conditioned when it achieves a success rate T2 per session of at least 70% for at least three consecutive sessions (chained or not).

According to a preferred embodiment, the model fish is deeply conditioned when it obtains a success rate T2 per session of at least 70% for one session, preferably for at least two consecutive sessions (chained or not), preferably for at least three consecutive sessions (chained or not); each session consisting of, or comprising, at least one repetition of 10 trials (performed in a chained manner or not). According to a particularly preferred embodiment, the model fish is deeply conditioned when it obtains a success rate T2 per session of at least 70% for at least three consecutive sessions (chained or not), each session consisting of a repetition of 10 trials performed in a chained manner.

The deep conditioning method can be implemented simultaneously on a group of individuals (belonging to the same genus of model fish or not, to the same species of model fish or not). Said group is defined in relation to the deep conditioning phase b) as previously described in relation to the conditioning phase a).

Thus, according to a first embodiment, the group of model fish is deeply conditioned when a predetermined number of individuals obtain a predetermined success rate T2 as defined above. The group of model fish is deeply conditioned when at least half (50%) of the individuals of the group obtain a predetermined success rate T2 as defined above. Preferably, the group of model fish is deeply conditioned when at least 55% of the individuals of the group obtain a predetermined success rate T2 as defined above, more preferably when at least 60% of the individuals of the group, more preferably when at least 65% of the individuals of the group, more preferably when at least 70% of the individuals of the group, more preferably when at least 75% of the individuals of the group, more preferably when at least 80% of the individuals of the group, more preferably when at least 85% of the individuals of the group, more preferably when at least 90% of the individuals of the group, more preferably when at least 95% of the individuals of the group, more preferably when 100% of the individuals of the group obtain a predetermined success rate T2 as defined above.

According to an embodiment, the step (b-iv) of detecting the response chosen by the model fish comprises detecting the passage and/or presence of said model fish in a response zone (or choice zone). The detection of the presence of the model fish in a response zone (or choice zone), as well as said response zone (or choice zone) are defined in relation to the deep conditioning step b) as previously described in relation to the conditioning step a). In particular, the detection of the presence of the model fish in a response zone (or choice zone), comprises for example the detection of the passage (or crossing) of said model fish through a response line (or border, limit, or boundary), said response line in relation to the deep conditioning phase b) being defined as previously indicated in relation to the conditioning phase a). In particular, said response line (and/or said response zone or choice zone) may consist of, or comprise, a response orifice (or opening, hole, or recess, cavity, or niche), said response orifice in relation to the deep conditioning phase b) being defined as previously described in relation to the conditioning phase a).

Advantageously, the step of detecting (b-iv) the response chosen by the model fish is performed by means of sensors, said sensors in relation to the deep conditioning phase b) being defined as previously indicated in relation to the conditioning phase a). Alternatively or in combination, the detection of the chosen response by the model fish (for example the detection of the passage of said model fish through a response zone) is visual, for example a detection directly by the experimenter.

It may be advantageous to detect the passage of the fish through a line that is not dedicated to the detection of the response chosen by the fish (for example the passage of the fish through an orifice that is not dedicated to the detection of the response chosen by the fish). Said line (for example comprising or consisting of an orifice) that is not dedicated to the detection of the response chosen by the fish is therefore called a tracking line (or monitoring line). The tracking line and the tracking orifice in relation to the deep conditioning phase b), are defined as previously indicated in relation to the conditioning phase a).

Alternatively or in combination, it may be advantageous to detect the movements and/or position of the model fish during all or part of a trial and/or a session and/or the deep conditioning phase b). In particular, such detection allows for the tracking (or monitoring) of the fish during the course of all or part of a trial and/or session and/or the deep conditioning procedure b). For example, the detection of the fish is carried out by means of sensors (for example by means of several sensors positioned in different zones (start zone, response zone), motion tracking camera(s), and/or a tracking or monitoring system. Alternatively or in combination, the detection can be visual, for example a detection directly by the experimenter.

According to an embodiment, the deep conditioning phase b) further comprises an additional step performed downstream of the detection step (b-v). Said additional step preferably comprises:
dispensing a reward to said model fish when it has chosen at least one of said response(s) identical to said instruction during said duration D_{rb}, said reward preferably comprising food suitable for said model fish; or
not delivering reward to said model fish when it has chosen at least one of said response(s) different from said instruction during said duration D_{rb} and, optionally, isolating and/or darkening said model fish, when it has chosen at least one of said response(s) different from said instruction (or optionally, when it has chosen no response) during said duration D_{rb}.

The present invention therefore relates to a method for obtaining deeply conditioned model fish, comprising a deep conditioning phase b); said deep conditioning phase being characterized in that it consists of at least one session of at least two consecutive trials; each trial comprising the steps consisting in:
(b-i) presenting an instruction to said conditioned model fish for a specified duration;
(b-ii) removing said instruction and then observing a delay of a predetermined duration D1, during which no instruction is presented to the model fish of step (b-i);
(b-iii) simultaneously presenting at least two distinct responses to the model fish of step (b-ii) for a duration D_{rb}, said at least two distinct responses comprising at least one response identical to said instruction and at least one response different from said instruction;
(b-iv) detecting the response chosen by the model fish of step (b-iii) from among said at least two distinct responses, during said duration D_{rb}; and
(b-v) dispensing a reward to said model fish when it has chosen at least one of said response(s) identical to said instruction during said duration D_{rb}, said reward preferably comprising food suitable for said model fish; or
not dispensing a reward to said model fish when it has chosen at least one of said response(s) different from said instruction during said duration D_{rb} and, optionally, isolating and/or darkening said model fish when it has chosen at least one of said response(s) different from said instruction (or optionally, when it has chosen no response) during said duration D_{rb}.

said model fish being deeply conditioned when it chooses at least one of said one or more identical responses to said instruction with a predetermined success rate T2 per session.

Thus, according to an embodiment, the method for obtaining a conditioned model fish according to the invention further comprises a deep conditioning phase b) characterized in that it consists of at least one session of at least two consecutive trials; each trial comprising the steps consisting in:
(b-i) presenting an instruction to said conditioned model fish for a specified duration;
(b-ii) removing said instruction and then observing a delay of a predetermined duration D1, during which no instruction is presented to the model fish of step (b-i);
(b-iii) simultaneously presenting at least two distinct responses to the model fish of step (b-ii) for a duration D_{rb}, said at least two distinct responses comprising at least one response identical to said instruction and at least one response different from said instruction;
(b-iv) detecting the response chosen by the model fish of step (b-iii) from among said at least two distinct responses, during said duration D_{rb}; and
(b-v) dispensing a reward to said model fish when it has chosen at least one of said response(s) identical to said instruction during said duration D_{rb}, said reward preferably comprising food suitable for said model fish; or
not dispensing a reward to said model fish when it has chosen at least one of said response(s) different from said instruction during said duration D_{rb} and, optionally, isolating and/or darkening said model fish when it has chosen at least one of said response(s) different from said instruction (or optionally, when it has chosen no response) during said duration D_{rb}.

said model fish being deeply conditioned when it chooses at least one of said one or more identical responses to said instruction with a predetermined success rate T2 per session.

The present invention therefore relates to a method for obtaining conditioned model fish, comprising a conditioning phase a); said conditioning phase being characterized in that it consists of at least one session of at least two consecutive trials; each trial comprising the steps consisting in:
(a-i) presenting an instruction to said model fish;
(a-ii) simultaneously presenting at least two distinct responses to the model fish of step (a-i) for a duration Dᵣₐ, said at least two distinct responses comprising at least one response identical to said instruction and at least one response different from said instruction;
(a-iii) detecting the response chosen by the model fish of step (a-ii) from among said at least two distinct responses during said duration Dᵣₐ; and, optionally,
(a-iv) dispensing a reward to said model fish when it has chosen at least one of said response(s) identical to said instruction during said duration Dᵣₐ, said reward preferably comprising food suitable for said model fish; or
not dispensing a reward to said model fish when it has chosen at least one of said response(s) different from said instruction during said duration Dᵣₐ and optionally isolating and/or darkening said model fish when it has chosen at least one of said response(s) different from said instruction (or optionally, when it has chosen no response) during said duration Dᵣₐ

said model fish being conditioned when it chooses at least one of said response(s) identical to said instruction with a predetermined success rate T1 per session;

said conditioning phase a) being followed by a deep conditioning phase b) characterized in that it consists of at least one session of at least two consecutive trials; each trial comprising the steps consisting in:
(b-i) presenting an instruction to said conditioned model fish for a specified duration;
(b-ii) removing said instruction and then observing a delay of a predetermined duration D1, during which no instruction is presented to the model fish of step (b-i);
(b-iii) simultaneously presenting at least two distinct responses to the model fish of step (b-ii) for a duration D_{rb}, said at least two distinct responses comprising at least one response identical to said instruction and at least one response different from said instruction; and
(b-iv) detecting the response selected by the model fish of step (b-iii) from said at least two distinct responses during said duration D_{rb}; and optionally
(b-v) dispensing a reward to said model fish when it has chosen at least one of said response(s) identical to said instruction during said duration D_{rb}, said reward preferably comprising food suitable for said model fish; or
not dispensing a reward to said model fish when it has chosen at least one of said response(s) different from said instruction during said duration D_{rb} and, optionally, isolating and/or darkening said model fish when it has chosen at least one of said response(s) different from said instruction (or optionally, when it has chosen no response) during said duration D_{rb}.

said model fish being deeply conditioned when it chooses at least one of said one or more identical responses to said instruction with a predetermined success rate T2 per session.

Advantageously, said reward of the deep conditioning phase b) is such as the reward defined in relation to the conditioning phase a). For example, the reward of the deep conditioning phase b) is dispensed by a reward dispenser. For example, the dispensing of said reward is triggered by the passage and/or detection of the presence of said model fish in a response zone, for example through a response line (comprising or consisting of a response orifice). Advantageously, said response zone, said response line and/or said response orifice is as defined above. In particular, said response line and/or said response orifice is dedicated to the detection of the response chosen by the fish. In particular, said response orifice may be an orifice of a reward dispenser or an orifice in a wall, such as a window, an opening, a hole or a recess, a cavity or a niche.

Advantageously, said reward is distributed to the model fish only when it has chosen at least one correct response during said duration D_{rb}. In this case, the model fish does not receive a reward when it has chosen at least one incorrect response during said duration D_{rb}. The correct response and incorrect response in relation to the deep conditioning phase b) are as defined above in relation to the conditioning phase a). In particular, according to an embodiment, for a given trial, the response is also said to be incorrect when no response choice by the model fish is detected at the expiration of the duration D_{rb}, during the detection step (b-iv). In other words, according to this embodiment, the response is also said to be incorrect when the model fish has not made any choice during said duration D_{rb}, during the detection step (b-iv). According to this embodiment, the response is thus said to be incorrect when said model fish chooses at least one of said response(s) different from the instruction of step (b-i), or when said fish has made no choice, during said duration of presentation of the responses D_{rb}. According to this embodiment, said duration D_{rb} represents the time given to the model fish to choose a response, during a trial. Said duration D_{rb} is as defined above.

According to an embodiment, the model fish is isolated when it has chosen at least one incorrect response during said duration D_{rb} (and/or the fish has made no choice during said duration D_{rb}, according to the embodiment), during a trial of the deep conditioning phase b). According to this isolation step, said model fish may for example be isolated (or enclosed, or confined) in a defined zone, for a determined duration Dᵢ. Advantageously, said determined duration Dᵢ during which the fish is isolated ranges from 5 seconds to 60 seconds, preferably from 10 seconds to 50 seconds, more preferably from 20 seconds to 40 seconds, more preferably from 25 seconds to 35 seconds, more preferably said duration is selected from 10 seconds, 15 seconds, 20 seconds, 25 seconds, 30 seconds, 35 seconds, 40 seconds and 45 seconds, and particularly preferably from about 30 seconds or 30 seconds. For example, the model fish is isolated in the start zone as defined above. Alternatively or in combination, the model fish is placed in darkness when it has chosen at least one incorrect response during said duration D_{rb} (and/or when the fish has made no choice during said duration D_{rb}, according to the embodiment), for said determined duration

According to an embodiment, said reward comprises or consists of food adapted to said model fish, such as commercially available dried and compacted feed granules, pellets or flakes (or a food), for example pellets of the Gemma® type, marketed by SKRETTING, such as Gemma Micro® (in particular Gemma Micro® 600). Advantageously, the reward comprises, or consists of, nutritionally poor feed (or food). More advantageously, the reward comprises, or consists of, palatable feed or a palatable food. More advantageously, the reward comprises, or consists of, palatable and nutritionally poor feed (or food). Advantageously, the reward comprises or consists of freeze-dried insect larvae, such as freeze-dried mosquito, fly or gnat larvae, or a mixture of freeze-dried mosquito and/or fly and/or gnat larvae (for example the freeze-dried insect larvae marketed by the firm JBL, in particular the NovoFil® range), or dried and compacted feed granules, pellets or flakes with a poor or depleted fat and/or protein and/or carbohydrate content (for example a pellet which provides less than 20%, preferably less than 15%, more preferably less than 10%, more preferably less than 5%, more preferably less than 3%, of the average daily amount of fat and/or protein and/or carbohydrate required by an adult model fish). In particular, the Inventors have shown that the success rate T2 is particularly optimal when the reward comprises freeze-dried insect larva. Preferably, the reward comprises or consists of freeze-dried mosquito larva.

According to an embodiment, the method of the invention further comprises a pre-conditioning (or pre-learning, or pre-education, or pre-training) phase pre-a), performed upstream of the conditioning phase a). Advantageously, said pre-conditioning phase comprises a step of adapting the model fish to the environment in which the conditioning phase a) and/or the deep conditioning phase b) will take place. The skilled person knows how to determine the steps comprised in said pre-conditioning phase. Advantageously, said pre-conditioning phase pre-a) in relation to the deep conditioning phase b) is as defined above in relation to the conditioning phase a).

The steps, instruction, response, trial, number of trials, session, number of sessions, reward, different durations (for example pause duration and interruption duration), detection step and detection, and model fish, predetermined success rate T3, of the pre-conditioning phase pre-a), in relation to the deep conditioning phase b), are defined as described above in relation to the conditioning phase a).

The present invention also relates to a deeply conditioned model fish obtainable by the method according to the invention as described above.

The present invention also relates to a deeply conditioned model fish obtained by the method according to the invention as described above. The present invention also relates to a deeply conditioned model fish directly obtained by the method according to the invention as described above.

### Device

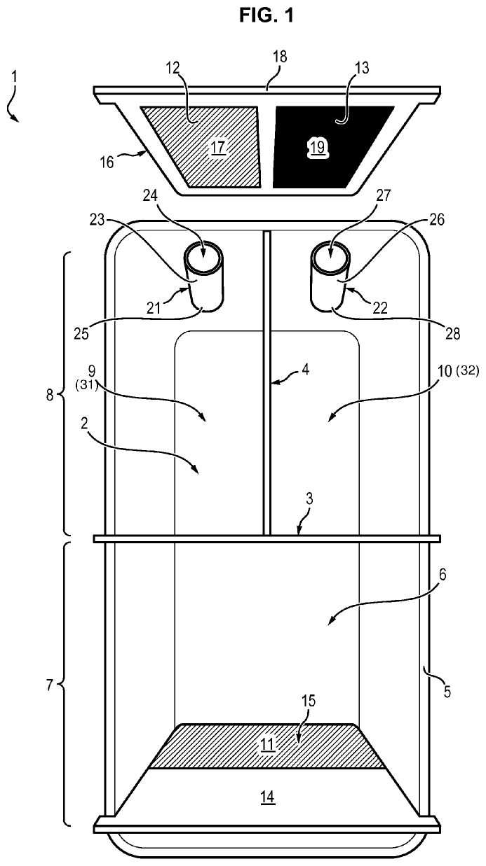

The device 1 illustrated in FIG. 1 comprises a tank 2, a first partition 3 and a second partition 4.

The tank 2 comprises a side wall 5 delimiting a cavity 6 suitable for containing water and in which the fish can be placed. The cavity 6 has a substantially rectangular shape. The side wall 5 may be formed of a transparent material, i.e., a material that allows light rays to pass in a range of vacuum wavelengths comprised between 380 nanometers (nm) and 780 nm.

The cavity 6 comprises a start zone 7 and a choice zone 8 (or response zone 8). The start zone 7 and the choice zone 8 are separated by the first partition 3. The first partition 3 runs parallel to a transverse plane of the cavity 6. The second partition 4 is disposed in the choice zone 8. The second partition 4 separates the choice zone 8 into a first compartment 9 and a second compartment 10. The second partition 4 extends perpendicular to the first partition 3. The second partition 4 extends parallel to a longitudinal plane of the cavity 6.

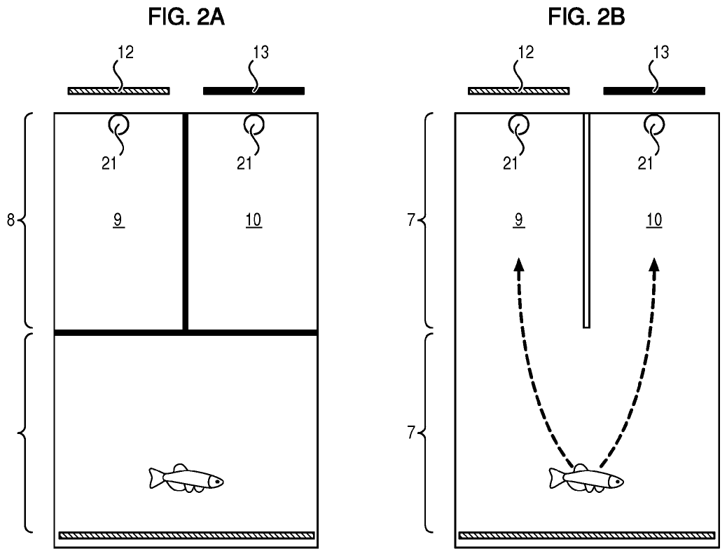

In the embodiment illustrated in FIG. 1, the first partition 3 is movably mounted with respect to the tank 2, between a closed position (schematically shown in FIG. 2A) in which the first partition 3 prevents a flow of fish between the start zone 7 and the choice zone 8, and an open position (schematically shown in FIG. 2B) in which the fish can flow between the start zone 7 and the choice zone 8. The first partition 3 can be made of an opaque material, i.e., a material that does not allow light rays to pass in a vacuum wavelength range comprised between 380 nanometers (nm) and 780 nm. In this way, when the first partition 3 is in the closed position, the fish in the start zone 7 cannot see the choice zone 8.

The second partition 4 may be mounted stationary or movable relative to the tank 2. The second partition 4 may also be made of an opaque material, i.e., a material that does not allow light rays to pass in a vacuum wavelength range comprised between 380 nanometers (nm) and 780 nm. In this way, when the fish is located in one of the compartments 9 and 10, the fish cannot see the other of the compartments.

The device 1 further comprises a first presentation element 11 for presenting an instruction in the start zone 7, a second presentation element 12 for presenting a first response in the first compartment 9 and a third presentation element 13 for presenting a second response in the second compartment 10.

In the example shown in FIG. 1, the instruction comprises a color. The first presentation element 11 comprises a first backing plate 14 and a first colored layer 15 extending over one side of the backing plate 14. The first presentation element 11 may be disposed in the cavity 6 of the tank 2, in the start zone 7. Alternatively, the first presentation element 11 may be disposed outside the tank 2, against the side wall 5, so that the first colored layer 15 is visible to the fish when the fish is in the start zone 7.

The first response comprises a first color and the second response comprises a second color, different from the first color. One of the first color and the second color is the same as the color of the instruction and the other of the first color and the second color is different from the color of the instruction. The second presentation element 12 comprises a second backing plate 16 and a second colored layer 17 of the first color extending over one side of the second backing plate 16. Similarly, the third presentation element 13 comprises a third backing plate 18 and a third colored layer 19 of the second color extending on one side of the third backing plate 18. The second presentation element 12 and the third presentation element 13 may be disposed within the tank 2, respectively in the first compartment 9 and in the second compartment 10 of the choice zone 8. Alternatively, the second presentation element 12 may be disposed outside the tank 2, against the side wall 5, so that the second colored layer 17 is visible to the fish when the fish is in the start zone 7, with the first partition 3 in the open position, and when the fish is in the first compartment 9. Similarly, the third presentation element 13 may be disposed outside the tank 2, against the side wall 5, so that the third colored layer 19 is visible to the fish when the fish is in the start zone 7, with the first partition 3 in the open position, and in the second compartment 10.

The presentation elements 11, 12, and 13 may be display panels for displaying the instruction, the first response, and the second response in a manner visible to the fish. The instruction, the first response, and the second response may be displayed manually, on screens, or projected (a color, shape, image, object, light signal, or any combination thereof). The device 1 further comprises a first reward dispenser 21 disposed in the first compartment 9 of the choice zone 8, and a second reward dispenser 22 disposed in the second compartment 10 of the choice zone 8.

The first reward dispenser 21 comprises a first tube 23. The first tube 23 has a first end having a first opening 24 and a second end having a second opening 25. The first tube 23 is disposed relative to the tank 2 such that the second end is flush with the surface of the water contained in the tank 2 in the first compartment 9.

The second reward dispenser 22 is identical to the first reward dispenser 21. The second reward dispenser 22 comprises a second tube 26. The second tube 26 has a first end having a first opening 27 and a second end having a second opening 28. The second tube 26 is disposed relative to the tank 2 such that the second end is flush with the surface of the water contained in the tank 2 in the second compartment 10.

Preferably, the first tube 21 and the second tube 22 are disposed in proximity to the second presentation element 12 and the third presentation element 13, respectively. The first tube 23 and the second tube 26 are disposed so that a portion of the side wall 5 of the tank 2 extends between the first tube 23 and the second presentation element 12, and another portion of the side wall 5 of the tank 2 extends between the second tube 26 and the third presentation element 13. In this manner, the fish passes in the vicinity of the second opening of the first tube 23 as it moves toward the second presentation element 12. Similarly, the fish passes near the second opening of the second tube 26 as it moves toward the third presentation element 13.

In use, the fish is initially positioned in the start zone 7, the first partition 3 being in the closed position. The instruction (i.e., the instruction color) is presented in the start zone 7 by the first presentation element 12. Then, after a predetermined time, the first partition 3 is moved from the closed position to the open position, so that the fish can move to the choice zone 8. The first response (i.e., the first response color) is presented in the first compartment 9 by the second presentation element 12. The second response (i.e., the second response color) is presented in the second compartment 10 by the third presentation element 13. Once the first partition 3 is open, the fish can enter either the first compartment 9 or the second compartment 10. If the fish enters the first compartment 9, it is considered to have chosen the first response. If the fish enters the second compartment 10, it is considered to have chosen the second response. Alternatively, another mode of detection can be used: the behavioral response is considered correct when the fish enters a response zone, which response zone can adopt different shapes and sizes, for example an orifice.

The device 1 may comprise one or more sensors 31, 32 suitable for detecting the response chosen by the fish. For example, the device 1 may comprise a first sensor 31 disposed in the first compartment 9 and suitable for detecting the presence of the fish in the first compartment 9, and a second sensor 32 disposed in the second compartment 10 and suitable for detecting the presence of the fish in the second compartment 10. The sensors 31, 32 may, for example, comprise motion sensors, such as sensors suitable for detecting infrared radiation, or a camera.

When the fish has chosen the correct response (i.e., the response that is identical to the instruction), a reward can be distributed. The reward is dispensed via the reward dispenser 21 or 22 located in the compartment 9 or 10 in which the fish is located. To this end, food is introduced into the tube 21 or 26 through the first opening 24 or 27. The food goes down the tube 21 or 26 to the surface of the water. The fish can pass its head through the second opening 25 or 28 to catch the food.

The same test can be repeated by changing the presentation elements.

In the example in which the presentation elements 11, 12 and 13 have just been described, other presentation elements may be used, such as display screens on which the instruction and responses are projected. The instruction can be a color, a shape, an image, an object, a light signal or a combination of these elements.

### Application Methods

Conditioned or deeply conditioned model fish are particularly suited for studies characterizing cognitive impairment or deficiency, for example, resulting from neuropsychiatric or neurological disease (such as neurodegenerative disease, addiction or brain injury); comprising, but not limited to, hyperactivity, autism, schizophrenia, Parkinson's disease, Alzheimer's disease, and brain injury; comprising effects on working memory and/or cognitive abilities and/or learning abilities in a model fish; or studies of brain regeneration capacity in a model fish; or studies to identify molecules, compounds, compositions or formulations that modify working memory and/or cognitive abilities and/or learning abilities in a model fish.

Thus, the present invention relates to a method of characterizing a cognitive impairment or deficiency, resulting for example from neuropsychiatric or neurological disease (such as neurodegenerative disease, addiction or brain injury), in a model fish, or to a method for studying the effect of a cognitive impairment or deficiency resulting for example from neuropsychiatric or neurological disease (such as neurodegenerative disease, addiction or brain injury), on working memory and/or cognitive abilities and/or learning abilities in a model fish, comprising the steps consisting in:
a) obtaining conditioned, or deeply conditioned, model fish by the method of the invention as described above, said conditioned, or deeply conditioned, model fish being model fish of said cognitive impairment or deficiency (for example model fish of said neuropsychiatric or neurological disease, such as model fish of said neurodegenerative disease, said addiction or said brain injury)
b) comparing the average time required to obtain the conditioned, or deeply conditioned, model fish of step a) with a reference average time and/or comparing the average success rate obtained by the conditioned model fish of step a) with a reference average success rate.

The average time required to obtain the conditioned, or deeply conditioned, model fish may, for example, consist of the sum of the times (in days, weeks or months) required for each individual (i.e., for each model fish of step a)) within a group of individuals conditioned simultaneously/in parallel or separately/independently, or deeply conditioned simultaneously/in parallel or separately/independently, to reach the predetermined success rate T1 or T2 as defined above, divided by the number of individuals considered. Alternatively, the average time required to obtain the conditioned model fish may for example consist of the sum of the times (in days, weeks or months) required for a group of individuals conditioned simultaneously/in parallel or separately/independently, or deeply conditioned simultaneously/in parallel or separately/independently, to reach the predetermined average success rate T1 or T2 as defined above, divided by the number of group of individuals considered. The reference average time may for example consist of an average time required to obtain a reference model fish, or a group of reference model fish, conditioned, or deeply conditioned, according to the method of the invention.

The average success rate is as defined above in relation to the method for obtaining conditioned, or deeply conditioned, model fish of the invention. The reference average success rate may, for example, consist of an average success rate obtained by a reference model fish, or a group of reference model fish, subjected to the method for obtaining conditioned, or deeply conditioned, model fish according to the invention.

Advantageously, the reference model fish belongs to the same genus and/or species as the model fish of step a). Said reference model fish is for example a healthy model fish. Said reference model fish is for example a model fish that is not a model fish of said cognitive impairment or deficiency, resulting for example from neuropsychiatric or neurological disease (such as neurodegenerative disease, addiction or brain injury). In this case, said cognitive impairment or deficiency, resulting for example from neuropsychiatric or neurological disease (such as neurodegenerative disease, addiction or brain injury) affects or inhibits working memory and/or cognitive abilities and/or learning abilities in a model fish if, as a result of the comparison of step b), the average time required to obtain the conditioned, or deeply conditioned, model fish of step a) is greater than the reference average time and/or the average success rate obtained by the conditioned, or deeply conditioned, model fish of step a) is less than the reference average success rate. Conversely, said cognitive impairment or deficiency, resulting for example from neuropsychiatric or neurological disease (such as neurodegenerative disease, addiction or brain injury) does not affect or inhibit working memory and/or cognitive abilities and/or learning abilities in a model fish if, as a result of the comparison of step b), the average time required to obtain the conditioned, or deeply conditioned, model fish of step a) is about equal to the reference average time and/or the average success rate obtained by the conditioned, or deeply conditioned, model fish of step a) is about equal to the reference average success rate.

According to an embodiment, the model fish of step a) is selected from a model fish of hyperactivity, autism, schizophrenia, Parkinson's disease, Alzheimer's disease, and brain injury.

The present invention also relates to a method for studying brain regeneration capacity in a model fish, comprising the steps consisting in:
a) obtaining conditioned, or deeply conditioned, model fish by the method of the invention as described above, said conditioned, or deeply conditioned, model fish being model fish of a cognitive impairment or deficiency, resulting for example from neuropsychiatric or neurological disease (such as neurodegenerative disease, addiction or brain injury)
b) implementing the method for obtaining conditioned or deeply conditioned model fish as described above on the conditioned or deeply conditioned model fish of step a), after an interval of a determined minimum duration, preferably ranging from 0.5 to 6 months, preferably from 1 to 5 months, more preferably from 2 to 4 months, more preferably from 3 months;
c) comparing the average success rate obtained by the conditioned, or deeply conditioned, model fish of step a) with the average success rate obtained by the conditioned, or deeply conditioned, model fish of step b).

According to an embodiment, said model fish exhibits brain regeneration capacity if, following the comparison of step c), the average success rate obtained by the conditioned, or deeply conditioned, model fish of step b) is greater than the average success rate obtained by the conditioned, or deeply conditioned, model fish of step a). Conversely, said model fish does not exhibit brain regeneration capacity if, as a result of the comparison of step c), the average success rate obtained by the conditioned, or deeply conditioned, model fish of step b) is less than or equal to the average success rate obtained by the conditioned, or deeply conditioned, model fish of step a).

The invention also relates to a method for characterizing working memory and/or cognitive abilities and/or learning abilities in a model fish, comprising the steps consisting in:
a) obtaining conditioned, or deeply conditioned, model fish by the method of the invention as described above;
b) determining the average time required to obtain the conditioned, or deeply conditioned, model fish of step a) and/or determining the average success rate obtained by the conditioned, or deeply conditioned, model fish of step a) and/or determining the maximum duration D1 applicable to the deeply conditioned model fish of step a); and optionally
c) comparing the average time of step b) with a reference average time and/or comparing the average success rate of step b) with a reference average success rate and/or comparing the maximum duration D1 of step b) with a reference maximum duration D1.

The average time required to obtain the conditioned, or deeply conditioned, model fish may, for example, consist of the sum of the times (in days, weeks or months) required for each individual (i.e., for each model fish of step a)) within a group of individuals conditioned simultaneously/in parallel or separately/independently, or deeply conditioned simultaneously/in parallel or separately/independently, to reach the predetermined success rate T1 or T2 as defined above, divided by the number of individuals considered.

Alternatively, the average time required to obtain the conditioned, or deeply conditioned, model fish may for example consist of the sum of the times (in days, weeks or months) required for a group of individuals conditioned simultaneously/in parallel or separately/independently, or deeply conditioned simultaneously/in parallel or separately/independently, to reach the predetermined average success rate T1 or T2 as defined above, divided by the number of the group of individuals considered. The reference average time may, for example, consist of an average time required to obtain a reference model fish, or a group of reference model fish, conditioned, or deeply conditioned, according to the method of the invention.

The average success rate is as defined above in relation to the conditioned, or deeply conditioned, model fish method of the invention. The reference average success rate may, for example, consist of an average success rate obtained by a reference model fish, or a group of reference model fish, subjected to the method for obtaining conditioned, or deeply conditioned, model fish according to the invention.

The maximum duration D1 applicable to the conditioned model fish of step a) is the maximum duration D1 for which the conditioned model fish of step a), subjected to the method for obtaining deeply conditioned model fish according to the invention, achieves a predetermined success rate T2 as defined above. Advantageously, the maximum duration D1 (or limit) represents the maximum working memory capacity (or limit) of the model fish. Thus, it is possible to gradually increase the duration D1 during different consecutive trials, for example in order to determine the maximum duration D1 (or limit duration D1) of the model fish considered under the conditions considered. The reference maximum duration D1 may, for example, consist of a maximum duration D1 obtained by a reference model fish, or a group of reference model fish, subjected to the method for obtaining deeply conditioned model fish according to the invention.

The reference model fish is for example a healthy model fish. Advantageously, the reference model fish belongs to the same genus and/or species as the model fish of step a). According to an embodiment, the model fish of step a) is a healthy model fish. According to an alternative embodiment, the model fish of step a) is selected from model fish of a cognitive impairment or deficiency, resulting for example from neuropsychiatric or neurological disease (such as neurodegenerative disease, addiction or brain injury), in particular selected from model fish of hyperactivity, autism, schizophrenia, Parkinson's disease, Alzheimer's disease, and brain injury. According to this alternative embodiment, the reference model fish is preferably a healthy model fish.

According to an embodiment, the model fish of step a) is a deeply conditioned model fish. According to this embodiment, the reference model fish may be a model fish conditioned according to the method of the invention, which has not been deeply conditioned. Advantageously, the reference model fish belongs to the same genus and/or species as the model fish of step a). Advantageously, the reference model fish is a healthy model fish if the model fish of step a) is a healthy model fish. Alternatively, if the model fish of step a) is chosen among model fish of a cognitive impairment or deficiency, resulting for example from neuropsychiatric or neurological disease (such as neurodegenerative disease, addiction or brain injury), in particular selected from model fish of hyperactivity, autism, schizophrenia, Parkinson's disease, Alzheimer's disease, and brain injury, then the reference model fish is a model fish of said cognitive impairment or deficiency.

The invention also relates to a method for identifying molecules, compounds, compositions or formulations that modify working memory and/or cognitive abilities and/or learning abilities in a model fish, or a method for screening molecules, compounds, compositions or formulations that modify working memory and/or cognitive abilities and/or learning abilities in a model fish.

In an embodiment, said method for identifying molecules, compounds, compositions or formulations that modify working memory and/or cognitive abilities and/or learning abilities in a model fish, comprises the steps consisting in:
a) bringing model fish into contact with said molecule, compound, composition or formulation;
b) implementing the method for obtaining conditioned, or deeply conditioned, model fish as described above on the model fish of step a);
c) comparing the average time required to obtain the conditioned, or deeply conditioned, model fish of step a) with a reference average time and/or comparing the average success rate obtained by the conditioned, or deeply conditioned, model fish of step a) with a reference average success rate.

According to this embodiment, said molecule, compound, composition or formulation restores and/or improves and/or positively modifies the working memory and/or cognitive abilities and/or learning abilities of said model fish if, as a result of the comparison of step c) the average time required to obtain the conditioned, or deeply conditioned, model fish of step b) is less than the reference average time and/or the average success rate obtained by the conditioned, or deeply conditioned, model fish of step b) is greater than the reference average success rate. Conversely, said molecule, compound, composition or formulation disrupts and/or inhibits and/or negatively affects and/or alters and/or negatively modifies the working memory and/or cognitive abilities and/or learning abilities of said model fish if, as a result of the comparison of step c), the average time required to obtain the conditioned, or deeply conditioned, model fish of step b) is greater than the reference average time and/or the average success rate obtained by the conditioned, or deeply conditioned, model fish of step b) is less than the reference average success rate.

In an alternative embodiment, said method for identifying molecules, compounds, compositions or formulations that modify working memory and/or cognitive abilities and/or learning abilities in a model fish, comprises the steps consisting in:
a) obtaining conditioned, or deeply conditioned, model fish by the method of the invention as described above, in the presence of said molecule, compound, composition or formulation;
b) comparing the average time required to obtain the conditioned, or deeply conditioned, model fish of step a) with a reference average time and/or comparing the average success rate obtained by the conditioned, or deeply conditioned, model fish of step a) with a reference average success rate.

According to this alternative embodiment, said molecule, compound, composition or formulation restores and/or improves and/or positively affects, and/or positively modifies the working memory and/or cognitive abilities and/or learning abilities of said model fish if, as a result of the comparison of step b), the average time required to obtain the conditioned, or deeply conditioned, model fish of step a) is less than the reference average time and/or the average success rate obtained by the conditioned, or deeply conditioned, model fish of step a) is greater than the reference average success rate. Conversely, said molecule, compound, composition or formulation disrupts and/or inhibits and/or negatively affects and/or alters and/or negatively modifies the working memory and/or cognitive abilities and/or learning abilities of said model fish if, as a result of the comparison of step b), the average time required to obtain the conditioned, or deeply conditioned, model fish of step a) is greater than the reference average time and/or the average success rate obtained by the conditioned, or deeply conditioned, model fish of step a) is less than the reference average success rate.

Regardless of these embodiments, this method also allows the study of the effect of molecules, compounds, compositions or formulations on working memory and/or cognitive abilities and/or learning abilities in a model fish.

The average time required to obtain the conditioned, or deeply conditioned, model fish may, for example, consist of the sum of the times (in days, weeks or months) required for each individual (i.e., for each model fish of step a)) within a group of individuals conditioned simultaneously/in parallel or separately/independently, or deeply conditioned simultaneously/in parallel or separately/independently, to reach the predetermined success rate T1 or T2 as defined above, divided by the number of individuals considered. Alternatively, the average time required to obtain the conditioned, or deeply conditioned, model fish may for example consist of the sum of the times (in days, weeks or months) required for a group of individuals conditioned simultaneously/in parallel or separately/independently, or deeply conditioned simultaneously/in parallel or separately/independently, to reach the predetermined average success rate T1 or T2 as defined above, divided by the number of the group of individuals considered. The reference average time may, for example, consist of an average time required to obtain a reference model fish, or a group of reference model fish, conditioned, or deeply conditioned, according to the method of the invention.

The average success rate is as defined above in relation to the conditioned, or deeply conditioned, model fish method of the invention. The reference average success rate may, for example, consist of an average success rate obtained by a reference model fish, or a group of reference model fish, subjected to the method for obtaining conditioned, or deeply conditioned, model fish of the invention.

Said reference model fish is for example a healthy model fish. Advantageously, the reference model fish is of the same genus and/or species as the model fish of step a). Advantageously, the reference model fish has not been contacted with said molecule, compound, composition or formulation. A reference model fish that has not been contacted with said molecule, said compound, said composition or said formulation is a “naive” fish.

According to an embodiment, the model fish of step a) is selected from model fish of a cognitive impairment or deficiency, resulting for example from neuropsychiatric or neurological disease (such as neurodegenerative disease, addiction or brain injury), in particular from model fish of hyperactivity, autism, schizophrenia, Parkinson's disease, Alzheimer's disease, and brain injury. In this case, the reference model fish may be a healthy model fish or a model fish of said cognitive impairment or deficiency. Advantageously, the reference model fish belongs to the same genus and/or species as the model fish of step a).

Advantageously, the reference model fish has not been brought into contact with said molecule, said compound, said composition or said formulation (“naive” reference model fish).

The invention also relates to a method for identifying molecules, compounds, compositions or formulations, or a method for screening molecules, compounds, compositions or formulations that modify working memory and/or cognitive abilities and/or learning abilities in a conditioned, or deeply conditioned, model fish according to the invention, comprising the steps consisting in:
a) providing conditioned, or deeply conditioned, model fish by the method for obtaining conditioned, or deeply conditioned, model fish of the invention as described above;
b) bringing the conditioned, or deeply conditioned, model fish of step a) into contact with said molecule, compound, composition or formulation;
c) implementing the method for obtaining conditioned, or deeply conditioned, model fish as described above on the model fish of step b);
d) comparing the average success rate obtained by the conditioned, or deeply conditioned, model fish of step a) with the average success rate obtained by the conditioned, or deeply conditioned, model fish of step c).

This method also makes it possible to study the effect of molecules, compounds, compositions or formulations on working memory and/or cognitive abilities and/or learning abilities in a model fish.

According to an embodiment, said molecule, compound, composition or formulation restores and/or improves and/or positively affects, and/or positively modifies the working memory and/or cognitive abilities and/or learning abilities of said model fish if, as a result of the comparison of step d), the average success rate obtained by the conditioned, or deeply conditioned, model fish of step c) is higher than the average success rate obtained by the conditioned, or deeply conditioned, model fish of step a). Conversely, said molecule, compound, composition or formulation disrupts and/or inhibits and/or negatively affects and/or alters and/or negatively modifies the working memory and/or cognitive abilities and/or learning abilities of said model fish if, as a result of the comparison of step d), the average success rate obtained by the conditioned, or deeply conditioned, model fish of step c) is lower than the average success rate obtained by the conditioned, or deeply conditioned, model fish of step a).

According to an embodiment, the model fish of step a) is selected from model fish of a cognitive impairment or deficiency, resulting for example from neuropsychiatric or neurological disease (such as neurodegenerative disease, addiction or brain injury), in particular from among model fish of hyperactivity, autism, schizophrenia, Parkinson's disease, Alzheimer's disease, and brain injury.

### Use

Conditioned or deeply conditioned model fish are particularly suited for studies characterizing cognitive impairment or deficiency, for example, resulting from neuropsychiatric or neurological disease (such as neurodegenerative disease, addiction or brain injury); including, but not limited to, hyperactivity, autism, schizophrenia, Parkinson's disease, Alzheimer's disease, and brain injury; in particular effects on working memory and/or cognitive abilities and/or learning abilities in a model fish; or studies of brain regeneration capacity in a model fish; or studies to identify molecules, compounds, compositions or formulations that modify working memory and/or cognitive abilities and/or learning abilities in a model fish.

Thus, the present invention relates to the use of a conditioned, or deeply conditioned, model fish obtainable by the method of the invention as described above, obtained by the method of the invention as described above, and/or directly obtained by the method of the invention as described above, to:
study and/or characterize a cognitive impairment or deficiency, resulting for example from neuropsychiatric or neurological disease (such as neurodegenerative disease, addiction or brain injury); including in particular hyperactivity, autism, schizophrenia, Parkinson's disease, Alzheimer's disease, and brain injury; in particular the effects on working memory and/or on cognitive abilities and/or on learning abilities in said model fish;
study the regenerative capacity of the brain in said model fish;
characterize working memory and/or cognitive abilities and/or learning abilities in a model fish; or
identify or screen molecules, compounds, compositions or formulations that modify working memory and/or cognitive abilities and/or learning abilities in a model fish.

The present invention further relates to the use/application of the method for obtaining a conditioned, or deeply conditioned, model fish according to the invention to
study and/or characterize a cognitive impairment or deficiency, resulting for example from neuropsychiatric or neurological disease (such as neurodegenerative disease, addiction or brain injury); including in particular hyperactivity, autism, schizophrenia, Parkinson's disease, Alzheimer's disease, and brain injury; in particular the effects on the working memory and/or on the cognitive abilities and/or on the learning abilities in said model fish;
study the regenerative capacity of the brain in said model fish;
characterize working memory and/or cognitive abilities and/or learning abilities in a model fish; or
identify or screen molecules, compounds, compositions or formulations that modify working memory and/or cognitive abilities and/or learning abilities in a model fish.

According to an embodiment of any one of the uses described above, said model fish is selected from model fish of a cognitive impairment or deficiency, resulting for example from neuropsychiatric or neurological disease (such as neurodegenerative disease, addiction or brain injury), in particular from model fish of hyperactivity, autism, schizophrenia, Parkinson's disease, Alzheimer's disease, and brain injury.

Advantageously, the uses described above are in accordance with the application methods as defined in the preceding section. Thus, the uses described above are implemented according to the application methods as defined in the preceding section.

### EXAMPLES

### Part A: Materials and Methods

### A.1 Fish Rearing

The fish are raised in an aquatic animal facility under controlled conditions. They are fed 2 to 3 times a day with gemma micro 300, the temperature is 28° C., the day/night cycle is 14 h of day and 10 h of night, with a continuous water change.

### A.2. Preparation of the Fish

A batch of 10 adult Danio rerio fish (3 to 12 months old) is placed in a behavior room. Each fish is isolated in a 6 L test aquarium, so there is no olfactory or chemical contact but visual contact persists. The temperature and the day/night cycle are the same as above. The water is changed manually once a week, and the feeding is done once a day with gemma micro 600. For 1 week the fish are put in habituation to become familiar with their new environment. During the weekend, the fish are deprived of food. This overall decrease in the amount of food compared to that provided in the animal facility will be important later on for the motivation of the fish to perform the task.

### A.3. Pre-Learning (Pre-Conditioning) Phase

The pre-learning (pre-conditioning) phase consists in conditioning a fish to collect a reward in a predefined zone (choice zone, response zone or reward zone, FIGS. 1 and 2). The reward is positioned in a food dispenser (for example a pipette). To take the reward, the animal must pass its head through the dispenser (FIGS. 1 and 2). In the following experiments, the reward is a piece of mosquito larva, a highly palatable food with low protein content so that the fish is not satiated.

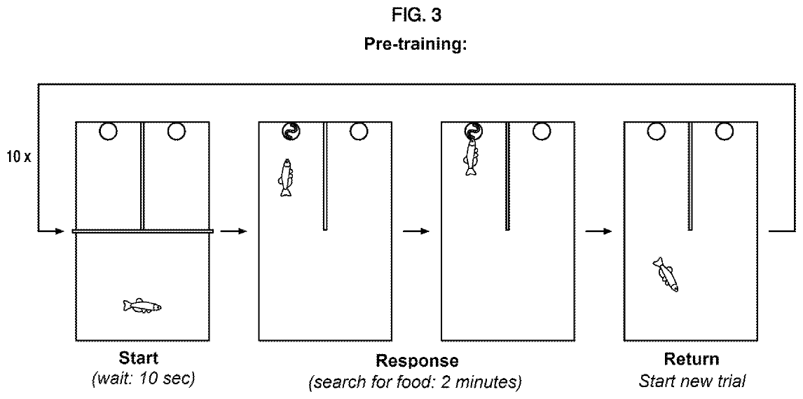

Day 1 and 2: At the beginning of a trial, the fish is placed in the start zone and the barrier between the start zone and the choice zone is closed for 10 s (FIG. 3). The barrier is then lifted. The fish can then go to the left or right reward zone, it has up to 2 min to do so (FIG. 3). If the fish does not move to a reward zone within the time limit, it is moved back to the start zone (for example with a net) and a new trial starts. Conversely, as soon as it approaches the reward zone, a reward is provided (FIG. 3) and it is allowed to return to the start zone. The barrier is closed and a new trial begins. Each session (series) consists of 10 consecutive trials, performed according to the procedure described above. One session (series) is performed per day. Therefore, 10 trials are performed per day.

This pre-learning phase allows the individual to become familiar with the reward zone so that it is no longer afraid of it. It also learns to approach the food dispenser (pipette): with each new attempt, the reward is deposited closer and closer to the dispenser, until it is deposited inside it. This step allows the correct behavioral response to be set: the fish enters a response zone, which can adopt different shapes and sizes, for example comprising, or consisting of an orifice, to which more complex conditions will be added later in the case of the tests used (SMTS, DMTS).

Days 3, 4 and 5: The pre-learning phase can optionally be extended to days 3, 4 or 5 if and only if some individuals have not learned to retrieve the reward from the reward dispenser. As soon as all fish have learned to retrieve the reward from the dispenser, the next step of the protocol is performed.

A fish is considered to be pre-conditioned when it obtains 70% success per session (a trial being considered successful when the fish takes the reward in the reward dispenser).

At the end of the 5^{th} day, the aquarium is cleaned and the fishes are fed with gemma 600 in order to have a protein contribution. During the weekend, the fish are deprived of food.

### A.4. Conditioning Phase

The learning phase of the task (conditioning phase) then begins. In precise scientific terms, the paradigm used is operant conditioning (active behavioral response to a presented stimulus). The task can consist of a “matching to sample” (MTS, or “simultaneous matching to sample” (SMTS)), according to the rule explained below. The fish must choose to go to the reward zone that shows an identical response to the instruction (or cue) presented in the start zone. In the case where the instruction is a color, the fish must choose to go to the reward zone that shows the same color as the cue presented in the start zone. It will be rewarded if and only if it chooses the same color as the cue presented (“sample”). In other words, the fish learns the rule: “the zone to choose is the one that shows the same color as the cue (“matching”)”.

### Description of a One-Session Trial in the Conditioning Phase:

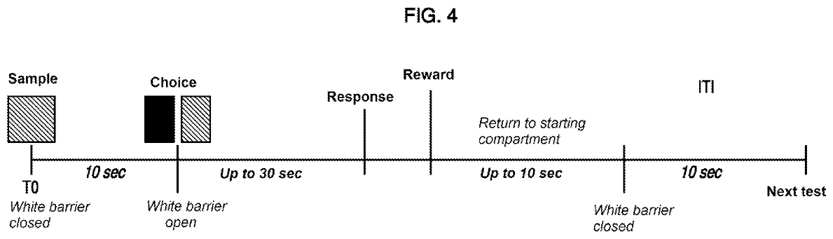

The course of a trial in the conditioning phase is summarized in FIG. 4.

During a trial, the fish is placed in the start zone for 10 s without cueing (FIG. 4). This period is called the intertrial interval (ITI).

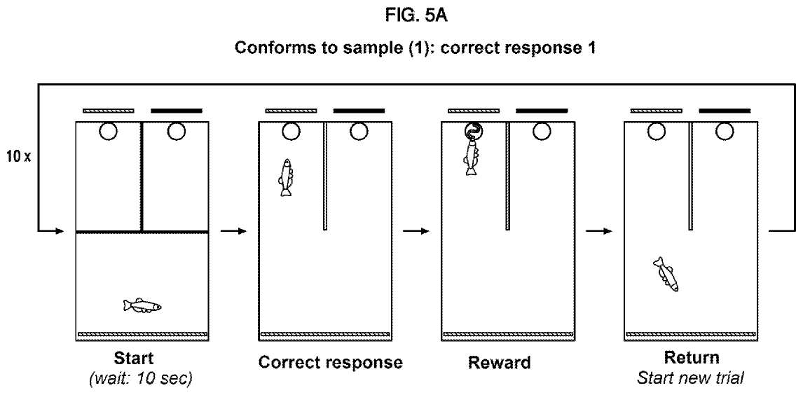

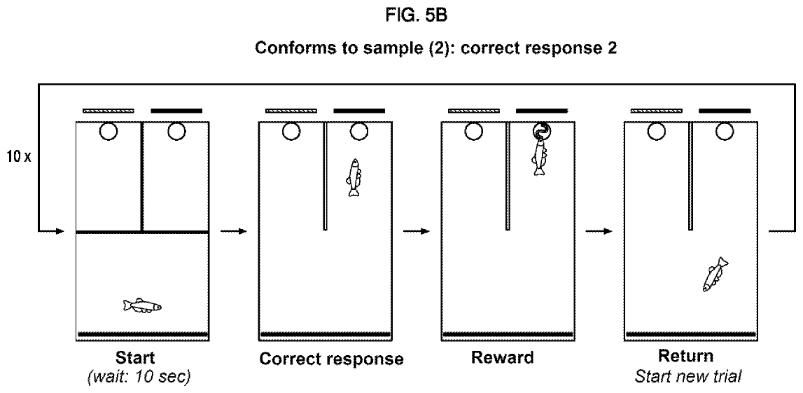

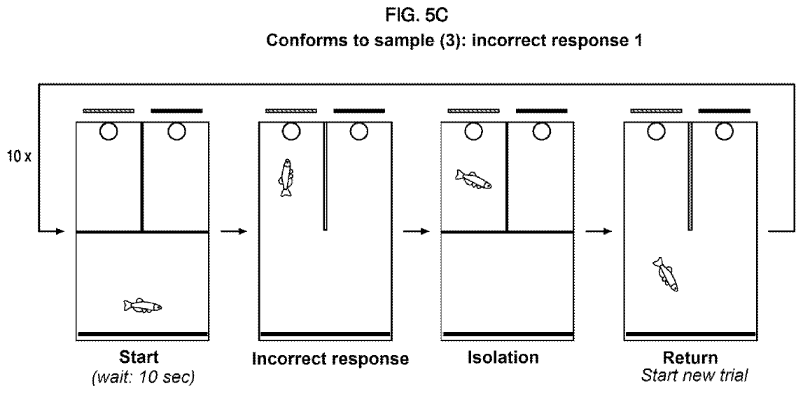

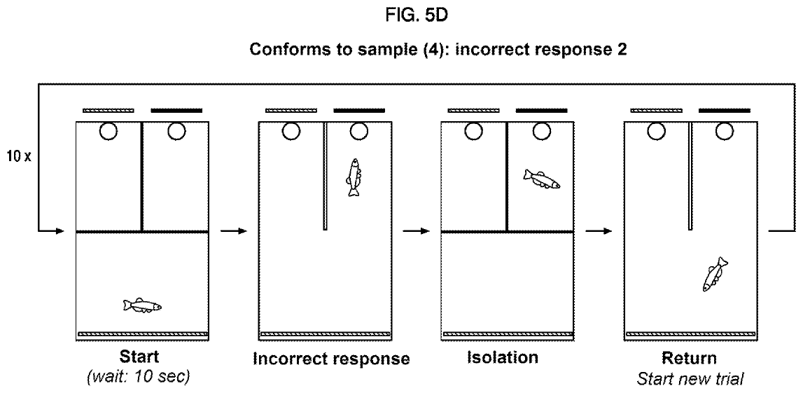

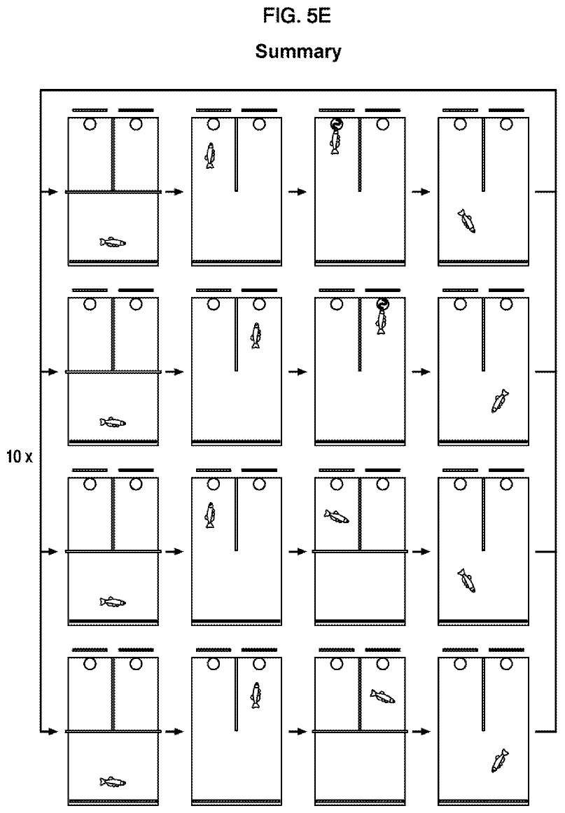

A colored cue (or instruction) in the form of a card is then presented in the start zone for 10 s (FIG. 5 A-D). After this time the barrier is opened and the fish has 30 s to choose a reward zone (FIG. 5 A-D). If the chosen zone is the correct response (color identical to the one shown in the start zone) then the fish gets a reward (FIG. 5 A-B). If the chosen zone is the “wrong” response (different color from the one shown in the start zone) then the fish is locked in the choice zone without a reward for 30 s (this is a mild punishment; FIG. 5 C-D). The colored cue is then removed. The fish then returns to the start zone and a new trial can begin.

Each session (series) consists of 10 consecutive trials, performed according to the procedure described above (FIG. 5 A-E). One session (series), which may or may not be consecutive is performed per day.

The number of sessions (or days of conditioning) required depends on the individual (the number of days of conditioning from 15 days to 1 month). The fish is considered to have acquired the conditioning when it obtains at least 70% correct responses per session for 3 consecutive sessions, carried out on 3 consecutive days.

### A.5. Deep Conditioning Phase

Once the conditioning phase is completed, the ability to retain information is tested with a deep conditioning phase (delayed matching to sample or (DMTS)). During this phase, the fish must remember the colored cue shown and choose the correct reward zone.

### Description of a One-Session Trial in the Deep Conditioning Phase:

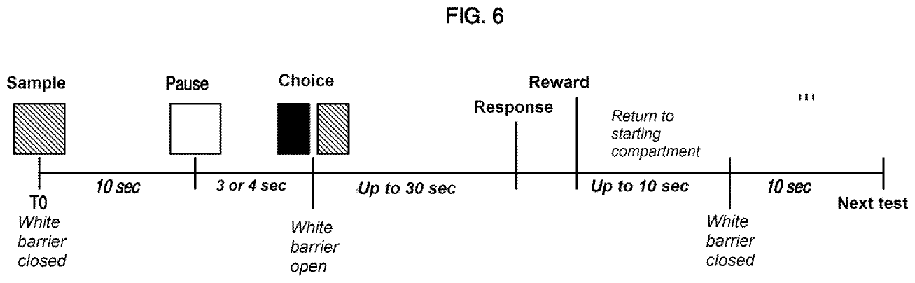

The course of a trial is summarized in FIG. 6.

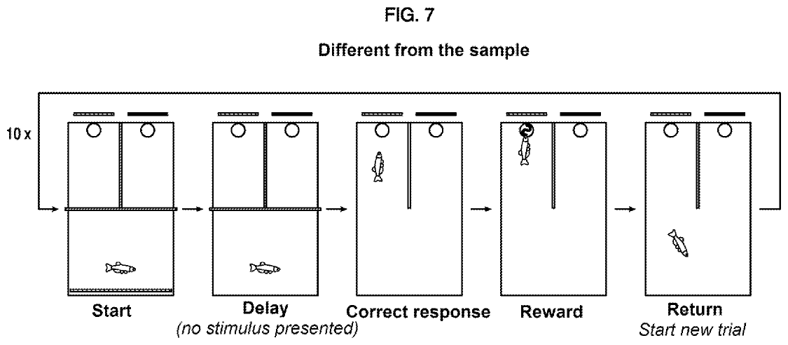

During a trial, the fish is in the start zone for 10 s without a cue (ITI, FIG. 6) a colored cue in the form of a card is presented in the start zone for 10 s (FIG. 7). The cue is then removed. The barrier is opened 3 sec later. The fish has 30 s to choose a reward zone (FIG. 7). If the chosen zone is “wrong” (different color than the one shown in the start zone) then the fish is locked in the choice zone without reward for 30 s (this is a mild punishment). If the chosen zone is the right one (same color as the one shown in the start zone) then the fish gets a reward. The fish then returns to the start zone. And a new trial can begin.

Each session (session) contains 10 trials and can be repeated over 3 consecutive days (one session per day) for more accurate results. If the fish obtains 70% correct responses per session, it is considered that it is capable of retaining the information and that the deep conditioning phase has been completed.

### Part B: Results

### Example B.1: Experiment 1

In this experiment, Gemma Micro 600 was used as a reward. These pellets are very nutritious and are commonly used in zebrafish housing facilities.

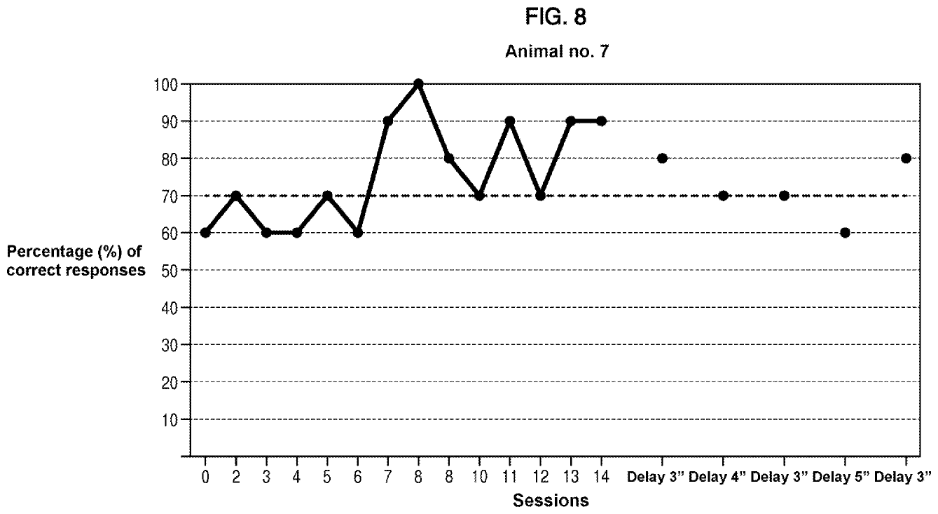

Table 1 below shows the percentage (%) of correct responses in each session (series) for each animal. FIG. 8 shows, in graphical form, this percentage for a typical fish (fish no. 7 in this experiment).

### B.1.1. Conditioning Phase or SMTS (“Simultaneous Matching-to-Sample”)

A batch of 10 adult zebrafish (Danio rerio) was subjected to the SMTS conditioning phase as described above (paragraph A.4).

During the tests, animal no. 6 exhibited “freezing” behavior (immobility) and was therefore excluded from the analysis. The other 9 animals reached the stable learning criterion (more than 70% correct responses per session over three consecutive sessions), at least once (Table 1 and FIG. 8).

In the first session (series), the rate of correct responses was random (30-60%). Two animals (no. 3 and 7) reached 70% correct responses on the second session (Table 1 and FIG. 8). Four fish (no. 1, 8, 9, and 10) reached the stable learning criterion in 7 sessions (series) or less (Table 1).

For practical reasons, the 14 sessions (series) do not correspond to 14 consecutive days. The 2^{nd} column “days” in Table 1 indicates the number of days since the first day of the experiment. For example, there was a pause of 11 days without a trial between the 8^{th} and 9^{th} session (series). It is interesting to note that 5 animals (no. 3, 4, 5, 7, and 9) achieved over 70% success even after 11 days without a trial between 2 sessions (Table 1 and FIG. 8).

These results show that all individuals reached 70% correct responses quickly. Certain individuals, however, had difficulty maintaining this level of performance in a continuous manner. It was considered that this could be due to a lack of motivation. In order to test this hypothesis, the frequency of the experiments was reduced: after the 6^{th} session, the following sessions were spaced at least two days apart. This allowed an overall improvement in the animals' performance (Table 1 and FIG. 8).

In conclusion, these results show for the first time that the zebrafish is able to learn SMTS. Moreover, this animal is able to learn SMTS despite irregular training days. Finally, these results show for the first time that the zebrafish is able to maintain a high response rate after several days without training.

### B.1.2. Deep Conditioning or Delayed Matching-to-Sample (DMTS) Phase

In this experiment, the DMTS test was performed after the 14 sessions of SMTS, on the batch of conditioned fish obtained after experiment B.1.1 described above. The delay time was 3 seconds for the first trial. 7 out of 9 fish were able to achieve more than 70% correct responses in the first session (Table 1, FIG. 8). The delay time was then increased to 4 seconds for the following session. Again 7 out of 9 fish achieved a correct response rate of more than 70%, which allowed testing a delay of 5 seconds for the next session. 2 subjects were able to achieve a correct response rate of at least 70%. The delay was reduced in the following session (3 seconds): all subjects were able to reach more than 70% correct response rate (Table 1, FIG. 8).

These data show for the first time that the zebrafish can perform DMTS deep conditioning with a delay of up to 4-5 seconds.

TABLE 1
Percentage of correct responses for each animal across sessions in Experiment 1.

| | Percentage (%) of correct responses | | | | | | | | | | |
|---|---|---|---|---|---|---|---|---|---|---|---|
| sessions | days | no. 1 | no. 2 | no. 3 | no. 4 | no. 5 | no. 7 | no. 8 | no. 9 | no. 10 | average |
| 1 | 1 | 40 | 30 | 50 | 40 | 40 | 60 | 40 | 50 | 40 | 43.3 |
| 2 | 2 | 60 | 40 | 70 | 30 | 60 | 70 | 50 | 60 | 60 | 55.6 |
| 3 | 3 | 50 | 60 | 80 | 60 | 60 | 60 | 70 | 40 | 60 | 60.0 |
| 4 | 4 | 70 | 80 | 60 | 80 | 80 | 60 | 80 | 70 | 60 | 71.1 |
| 5 | 5 | 80 | 60 | 60 | 60 | 60 | 70 | 80 | 70 | 70 | 67.8 |
| 6 | 8 | 80 | 40 | 70 | 60 | 80 | 60 | 70 | 100 | 100 | 73.3 |
| 7 | 10 | 60 | 60 | 90 | 70 | 40 | 90 | 90 | 50 | 100 | 72.2 |
| 8 | 12 | 80 | 70 | 60 | 50 | 60 | 100 | 70 | 90 | 60 | 71.1 |
| 9 | 23 | 60 | 60 | 80 | 80 | 80 | 80 | 60 | 70 | 60 | 70.0 |
| 10 | 25 | 70 | 90 | 60 | 50 | 80 | 70 | 80 | 100 | 80 | 75.6 |
| 11 | 29 | 60 | 80 | 90 | 80 | 70 | 90 | 90 | 80 | 90 | 81.1 |
| 12 | 32 | 80 | 60 | 70 | 80 | 70 | 70 | 60 | 70 | 60 | 68.9 |
| 13 | 36 | 60 | 90 | 70 | 80 | 100 | 90 | 90 | 80 | 70 | 81.1 |
| 14 | 39 | 90 | 80 | 100 | 70 | 70 | 90 | 90 | 90 | 80 | 84.4 |
| delay 3″ | 43 | 50 | 80 | 60 | 90 | 80 | 80 | 90 | 70 | 70 | 74.4 |
| delay 4″ | 46 | 50 | 70 | 70 | 80 | 90 | 70 | 80 | 80 | 60 | 72.2 |
| delay 3″ | 52 | 80 | 80 | 80 | 60 | 90 | 70 | 70 | 50 | 60 | 71.1 |
| delay 5″ | 59 | 30 | 60 | 70 | 40 | 100 | 60 | 30 | 30 | 40 | 51.1 |
| delay 3″ | 61 | 90 | 80 | 90 | 80 | 80 | 80 | 80 | 90 | 80 | 83.3 |

### Example B.2: Experiment 2

A decrease in motivation, probably related to satiety, was observed in Experiment 1, so the food reward was changed for Experiment 2. Dried mosquito larvae were cut into small pieces, and one piece at a time was given as a reward.

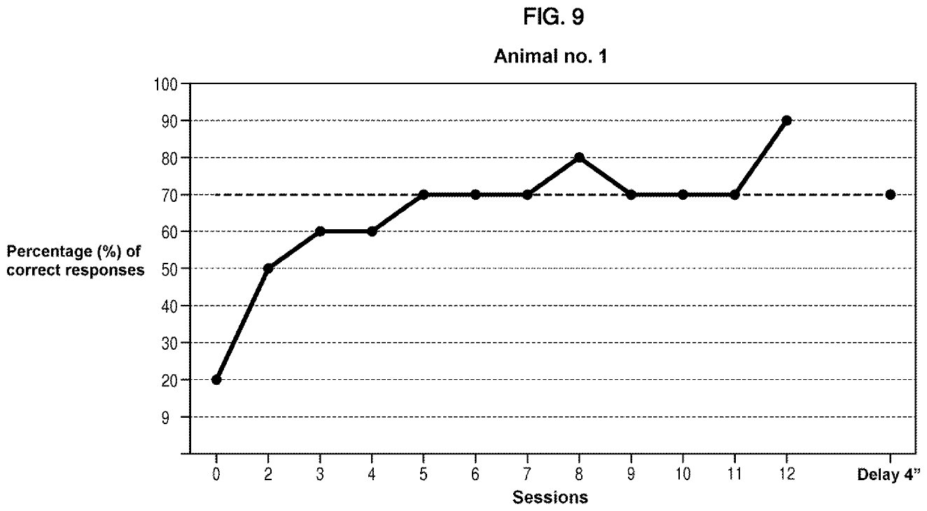

Table 2 shows the percentage (%) of correct responses in each session for each animal. FIG. 9 shows, in graphical form, this percentage for a typical fish, fish no. 1.

### B.2.1. Simultaneous Matching-to-Sample (SMTS)

Two individuals (no. 6 and no. 8) achieved a 70% correct response rate in the first session and maintained this level of performance throughout the experiment (Table 2). The remaining subjects started with near random performance, but all animals achieved a 70% correct response rate at least once during the first 6 sessions (Table 2 and FIG. 9). Of the 9 animals, 8 (with the exception of no. 10) achieved the stable learning criterion (70% success per session over 3 consecutive sessions) at least once by the end of the 12^{th} session. The level of performance was well maintained: certain individuals (no. 1) maintained a correct response level of more than 70% for 8 continuous sessions (Table 2 and FIG. 9).

Thus, the piece of mosquito larva as a reward made it possible to obtain better results than Gemma Micro 600, under the conditions tested.

### B.2.2. Delayed Matching-to-Sample (DMTS)

The DMTS test with 4 seconds of delay was performed after the majority of the animals reached the stable learning criterion in the MTS conditioning phase.

The results show that at least half of the individuals are able to perform DMTS with a success rate of at least 70%, from the first session (Table 2 and FIG. 9).

TABLE 2
Percentage of correct responses for each animal across sessions in Experiment 2.

| | | Percentage (%) of correct responses | | | | | | | | | |
|---|---|---|---|---|---|---|---|---|---|---|---|
| sessions | days | no. 1 | no. 2 | no. 3 | no. 4 | no. 5 | no. 6 | no. 7 | no. 8 | no. 10 | average |
| 1 | 1 | 20 | 50 | 50 | 40 | 60 | 70 | 40 | 70 | 50 | 50.0 |
| 2 | 2 | 50 | 40 | 50 | 80 | 60 | 70 | 60 | 50 | 60 | 57.8 |
| 3 | 3 | 60 | 40 | 50 | 50 | 60 | 70 | 50 | 90 | 50 | 57.8 |
| 4 | 4 | 60 | 60 | 80 | 90 | 70 | 60 | 80 | 80 | 60 | 71.1 |
| 5 | 5 | 70 | 70 | 50 | 60 | 70 | 70 | 70 | 30 | 50 | 60.0 |
| 6 | 8 | 70 | 50 | 60 | 70 | 80 | 80 | 70 | 30 | 70 | 64.4 |
| 7 | 9 | 70 | 70 | 70 | 80 | 50 | 60 | 70 | 70 | 90 | 70.0 |
| 8 | 11 | 80 | 90 | 80 | 70 | 40 | 70 | 50 | 90 | 50 | 68.9 |
| 9 | 12 | 70 | 80 | 60 | 70 | 50 | 90 | 70 | 80 | 50 | 68.9 |
| 10 | 16 | 70 | 70 | 80 | 70 | 60 | 70 | 70 | 70 | 60 | 68.9 |
| 11 | 17 | 70 | 70 | 100 | 80 | 80 | 80 | 90 | 100 | 80 | 83.3 |
| 12 | 23 | 90 | 70 | 90 | 80 | 100 | 90 | 70 | 90 | 60 | 82.2 |
| delay 4″ | 24 | 70 | 50 | 90 | 50 | 60 | 90 | 80 | 80 | 60 | 70.0 |

### REFERENCES

Arthur and Levin, 2001, Anim. Cogn., 4:125-131;
Bloch et al., 2019, Behavioural Brain Research, 370;
Cotwill et al., 2005, Behav Processes, August 31; 70(1):19-31;
Gierszewski et al., 2013, PLoS One 8(2):e57363;
Goldman and Shapiro, 1979, J. of the Exp. Analysis of Behaviour, March; 31(2):259-266;
Schluessel et al., 2012, Anim. Cogn., July; 15(4):525-37.
Schuster and Amtsfeld, 2002, J Exp Biol., February; 205(Pt 4):549-57;
Sovrano and Bisazza, 2009, Anim Cogn., 2008 January; 11(1):161-6;
Wyzisk and Neumeyer, 2007, Vis Neurosci., May-June; 24(3):291-8.

1. A method for obtaining conditioned model fish comprising a conditioning phase a), wherein the conditioning phase consists essentially of at least one session of at least two consecutive trials;
each trial comprising the steps consisting essentially in:
(a-i) presenting an instruction to said model fish;
(a-ii) simultaneously presenting at least two distinct responses to the model fish of step (a-i) for a duration Dᵣₐ, said at least two distinct responses comprising at least one response identical to said instruction and at least one response different from said instruction; and
(a-iii) detecting the response chosen by the model fish of step (a-ii) among said at least two distinct responses, during said duration Dᵣₐ, for example by means of sensors;
said model fish being conditioned when it chooses at least one of said response(s) identical to said instruction with a predetermined success rate T1 per session.

2. The method according to claim 1, further comprising a deep conditioning phase b) consisting essentially of at least one session of at least two consecutive trials;
each trial comprising the steps consisting essentially in:
(b-i) presenting an instruction to said conditioned model fish for a specified duration;
(b-ii) removing said instruction and then observing a delay of a predetermined duration D1, during which no instruction is presented to the model fish of step (b-i);
(b-iii) simultaneously presenting at least two distinct responses to the model fish of step (b-ii) for a duration D_{rb}, said at least two distinct responses comprising at least one response identical to said instruction and at least one response different from said instruction; and
(b-iv) detecting the response chosen by the model fish of step (b-iii) among said at least two distinct responses, during said duration D_{rb}, for example by means of sensors;
said model fish being deeply conditioned when it chooses at least one of said one or more identical responses to said instruction with a predetermined success rate T2 per session.

3. The method according to claim 2, wherein the duration D1 of said delay ranges from 0.5 to 10 seconds.

4. The method according to claim 1, wherein said success rate T1, and/or said success rate T2, is at least 70%.

5. The method according to claim 4, wherein said success rate T1 is achieved for at least two consecutive sessions, said success rate T1 preferably being achieved for at least two consecutive sessions as early as the fifteenth session performed.

6. The method according to claim 1, wherein said duration Dᵣₐ and/or said duration D_{rb}, ranges from 1 second to 300 seconds.

7. The method according to claim 1, wherein said instruction and/or said at least one response is a visual stimulus.

8. The method according to claim 1, wherein the step of detecting the response chosen by the model fish comprises detecting the passage of said model fish through a response zone.

9. The method according to claim 1, comprising an additional step (a-iv) and/or (b-v), said additional step comprising:
dispensing a reward to said model fish when it has chosen at least one of said response(s) identical to said instruction during said duration Dᵣₐ, and/or during said duration D_{rb}; or
not dispensing a reward to said model fish when it has chosen at least one of said response(s) different from said instruction (or optionally, when it has chosen no response) during said duration Dᵣₐ, and/or during said duration D_{rb}, and optionally, isolating and/or putting in the dark said model fish, when it has chosen at least one of said response(s) different from said instruction (or optionally, when it has chosen no response) during said duration Dᵣₐ, and/or during said duration D_{rb}.

10. The method according to claim 1, wherein said model fish is selected from model fish belonging to the genera Danio sp., Pseudotropheus sp., Gnathonemus sp., Pomacentrus sp., Phoxinus sp., Xenotoca sp., Astronotus sp., Oryzias sp, Nothobranchius sp., Astyanax sp. and Carassius sp.

11. A conditioned, or deeply conditioned, model fish obtainable by the method as defined in claim 1.

12. A device for obtaining conditioned model fish (1), comprising:
a tank (2) comprising a wall (5) delimiting a cavity (6) suitable for containing water for the fish, the cavity (6) comprising a start zone (7) and a choice zone (8),
a first partition (3) movable between a closed position in which the first partition (3) prevents movement of the model fish between the start zone (7) and the choice zone (8), and an open position in which the first partition (3) allows movement of the model fish between the start zone (7) and the choice zone (8),
a second partition (4) separating the choice zone (8) into a first compartment (9) and a second compartment (10), and
a first presentation element (11) for presenting an instruction in the start zone (7), a second presentation element (12) for presenting a first response in the first compartment (9) and a third presentation element (13) for presenting a second response in the second compartment (10).

13. The device according to claim 12, wherein the first presentation element comprises a first display panel, on which the instruction appears, the second presentation element comprises a second display panel, on which the first response appears and the third presentation element comprises a third display panel, on which the second response appears.

14. The device according to claim 12, wherein one of the first response and the second response is identical to the instruction and the other of the first response and the second response is different from the instruction.

15. The device according to claim 12, wherein the instruction is a color, and one of the first response and the second response is a color identical to the instruction color and the other of the first response and the second response is a different color than the instruction color.

16. The device according to claim 12, wherein the first movable partition is opaque to light radiation in a vacuum wavelength range comprised between 380 to 780 nanometers.

17. Method for characterizing a cognitive impairment or deficiency; or for characterizing working memory and/or cognitive abilities and/or learning abilities in a model fish; or for identifying molecules, compounds, compositions or formulations that modify working memory and/or cognitive abilities and/or learning abilities in a model fish, using a conditioned model fish obtainable by the method as defined in claim 1.

18. The method according to claim 17, wherein said model fish is selected from model fish of hyperactivity, autism, schizophrenia, Parkinson's disease, Alzheimer's disease and brain injury.
